SEQUENCE LISTING (53 sequences)

SEQ ID NO: 1 - DNA, 22 bp, Artificial Sequence
  Synthetic F4/80 forward primer
ctttggctatgggcttccagtc

SEQ ID NO: 2 - DNA, 24 bp, Artificial Sequence
  Synthetic F4/80 reverse primer
gcaaggaggacagagtttatcgtg

SEQ ID NO: 3 - DNA, 20 bp, Artificial Sequence
  Synthetic Arg1 forward primer
cacagtctggcagttggaag

SEQ ID NO: 4 - DNA, 21 bp, Artificial Sequence
  Synthetic Arg1 reverse primer
gggagtgttgatgtcagtgtg

SEQ ID NO: 5 - DNA, 23 bp, Artificial Sequence
  Synthetic Mgl1 forward primer
tgagaaaggctttaagaactggg

SEQ ID NO: 6 - DNA, 21 bp, Artificial Sequence
  Synthetic Mgl1 reverse primer
gaccacctgtagtgatgtggg

SEQ ID NO: 7 - DNA, 30 bp, Artificial Sequence
  Synthetic IL1b forward primer
aatctatacctgtcctgtgtaatgaaagac

SEQ ID NO: 8 - DNA, 22 bp, Artificial Sequence
  Synthetic IL1b reverse primer
ggtattgcttgggatccacact

SEQ ID NO: 9 - DNA, 22 bp, Artificial Sequence
  Synthetic IL10 forward primer
ctggacaacatactgctaaccg

SEQ ID NO: 10 - DNA, 22 bp, Artificial Sequence
  Synthetic IL10 reverse primer
gggcatcacttctaccaggtaa

SEQ ID NO: 11 - DNA, 20 bp, Artificial Sequence
  Synthetic IL6 forward primer
ctctgggaaatcgtggaaat

SEQ ID NO: 12 - DNA, 20 bp, Artificial Sequence
  Synthetic IL6 reverse primer
ccagtttggtagcatccatc

SEQ ID NO: 13 - DNA, 22 bp, Artificial Sequence
  Synthetic FAS forward primer
tcctggaacgagaacacgatct

SEQ ID NO: 14 - DNA, 23 bp, Artificial Sequence
  Synthetic FAS reverse primer
gagacgtgtcactcctggacttg

SEQ ID NO: 15 - DNA, 20 bp, Artificial Sequence
  Synthetic ACC1 forward primer
cgctcgtcaggttcttattg

SEQ ID NO: 16 - DNA, 20 bp, Artificial Sequence
  Synthetic ACC1 reverse primer
tttctgcaggttctcaatgc

SEQ ID NO: 17 - DNA, 21 bp, Artificial Sequence
  Synthetic ACC2 forward primer
cgctcaccaacagtaaggtgg

SEQ ID NO: 18 - DNA, 19 bp, Artificial Sequence
  Synthetic ACC2 reverse primer
gcttggcagggagttcctc

SEQ ID NO: 19 - DNA, 19 bp, Artificial Sequence
  Synthetic SCD1 forward primer
acgccgaccctcacaattc

SEQ ID NO: 20 - DNA, 21 bp, Artificial Sequence
  Synthetic SCD1 reverse primer
cagttttccgcccttctcttt

SEQ ID NO: 21 - DNA, 20 bp, Artificial Sequence
  Synthetic Srebp1c forward primer
ggagccatggattgcacatt

SEQ ID NO: 22 - DNA, 20 bp, Artificial Sequence
  Synthetic Srebp1c reverse primer
gcttccagagaggaggccag

SEQ ID NO: 23 - DNA, 20 bp, Artificial Sequence
  Synthetic AOX forward primer
cagacagagatgggtcatgg

SEQ ID NO: 24 - DNA, 20 bp, Artificial Sequence
  Synthetic AOX reverse primer
atgaactcttgggtcttggg

SEQ ID NO: 25 - DNA, 20 bp, Artificial Sequence
  Synthetic MCAD forward primer
tttcgaagacgtcagagtgc

SEQ ID NO: 26 - DNA, 20 bp, Artificial Sequence
  Synthetic MCAD reverse primer
tgcgactgtaggtctggttc

SEQ ID NO: 27 - DNA, 20 bp, Artificial Sequence
  Synthetic BSEP forward primer
gaaacgacggcgttcgtggg

SEQ ID NO: 28 - DNA, 23 bp, Artificial Sequence
  Synthetic BSEP reverse primer
gccatccagagtcaccatgcctt

SEQ ID NO: 29 - DNA, 21 bp, Artificial Sequence
  Synthetic PLTP forward primer
cgccccacgtgaccacactac

SEQ ID NO: 30 - DNA, 24 bp, Artificial Sequence
  Synthetic PLTP reverse primer
ggccttcctgcttcaccagatcca

SEQ ID NO: 31 - DNA, 25 bp, Artificial Sequence
  Synthetic OATP1 forward primer
cagattcaggcacatttacctgggg

SEQ ID NO: 32 - DNA, 23 bp, Artificial Sequence
  Synthetic OATP1 reverse primer
tgtctggagagtggatgtcgcca

SEQ ID NO: 33 - DNA, 24 bp, Artificial Sequence
  Synthetic LXRa forward primer
aagctggtgagcctccgtactttg

SEQ ID NO: 34 - DNA, 24 bp, Artificial Sequence
  Synthetic LXRa reverse primer
gcgaaacagtcactcgtggacatc

SEQ ID NO: 35 - DNA, 21 bp, Artificial Sequence
  Synthetic FXRa1/2 forward primer
cagccaccggctgtcaggatt

SEQ ID NO: 36 - DNA, 19 bp, Artificial Sequence
  Synthetic FXRa1/2 reverse primer
ggcctgccgcgtgttctgt

SEQ ID NO: 37 - DNA, 23 bp, Artificial Sequence
  Synthetic FXRa3/4 forward primer
ttgggcactcccatttacaggct

SEQ ID NO: 38 - DNA, 21 bp, Artificial Sequence
  Synthetic FXRa3/4 reverse primer
ggcctgccgcgtgttctgtta

SEQ ID NO: 39 - DNA, 21 bp, Artificial Sequence
  Synthetic SHP forward primer
cgatcctcttcaacccagatg

SEQ ID NO: 40 - DNA, 21 bp, Artificial Sequence
  Synthetic SHP reverse primer
agggctccaagacttcacaca

SEQ ID NO: 41 - DNA, 20 bp, Artificial Sequence
  Synthetic 36B4 forward primer
tcatccagcaggtgtttgac

SEQ ID NO: 42 - DNA, 20 bp, Artificial Sequence
  Synthetic 36B4 reverse primer
tacccgatctgcagacacac

SEQ ID NO: 43 - DNA, 36 bp, Artificial Sequence
  Synthetic Mut1 forward primer
ccagcttactaggaattctggcattaagaactttcc

SEQ ID NO: 44 - DNA, 36 bp, Artificial Sequence
  Synthetic Mut1 reverse primer
ggaaagttcttaatgccagaattcctagtaagctgg

SEQ ID NO: 45 - DNA, 31 bp, Artificial Sequence
  Synthetic Mut2 forward primer
ggcattaagaactttctagataaacatttac

SEQ ID NO: 46 - DNA, 31 bp, Artificial Sequence
  Synthetic Mut2 reverse primer
gtaaatgtttatctagaaagttcttaatgcc

SEQ ID NO: 47 - DNA, 33 bp, Artificial Sequence
  Synthetic Mut3 forward primer
ccgtaatgaagttaacgcgtaaaccacacaacc

SEQ ID NO: 48 - DNA, 33 bp, Artificial Sequence
  Synthetic Mut3 reverse primer
ggttgtgtggtttacgcgttaacttcattacgg

SEQ ID NO: 49 - DNA, 142 bp, Homo sapiens
tttccagcttactaggcaatttggcattaagaactttcctcataaacatttacaaatattctgctccgtaatgaagttaatcagtaaaccacacaacctctgtcctctctcactccttacctagtctcattttcagtggctg

SEQ ID NO: 50 - DNA, 143 bp, Rattus rattus
tttccagcttactaggcaatttggcattgaggacttttctcataaacatttacaaatattccgctcagcaatggagttaatcagtaaaccacaccaacctctggcccctgtcacttcttacctcggctccttctcagttgccg

SEQ ID NO: 51 - DNA, 143 bp, Mus musculus
tttccagcttactaggcaatttggcattgaggacttttctcataaacatttacaaatattccgctcagcgatggggttaatcagtaaaccacaccaacctctggcccctgtcgcttcttacctcggctccttctcagttgccg

SEQ ID NO: 52 - DNA, 11 bp, Artificial Sequence
  Synthetic DNA
ttaatcagtaa

SEQ ID NO: 53 - DNA, 11 bp, Artificial Sequence
  Synthetic DNA
ttaacgcgtaa

(19) United States
(12) Patent Application Publication
(10) Pub. No.: US 2014/0315781 A1
(43) Pub. Date: Oct. 23, 2014

(54) METHODS OF TREATING FATTY LIVER DISEASE WITH HELMINTH-DERIVED GLYCAN-CONTAINING COMPOUNDS

(71) Applicants: Chih-hao Lee, Boston, MA (US); Prerna Bhargava, Woodbridge, CT (US); Donald A. Harn, Athens, GA (US)

(72) Inventors: Chih-hao Lee, Boston, MA (US); Prerna Bhargava, Woodbridge, CT (US); Donald A. Harn, Athens, GA (US)

(73) Assignee: PRESIDENT AND FELLOWS OF HARVARD COLLEGE, Cambridge, MA (US)

(21) Appl. No.: 14/346,613

(22) PCT Filed: Sep. 21, 2012
(86) PCT No.: PCT/US2012/056595

§ 371 Date: Mar. 21, 2014

Related U.S. Application Data
(60) Provisional application No. 61/538,629, filed on Sep. 23, 2011.

(51) Int. Cl. A61K 31/726; A61K 47/48

(52) U.S. Cl. CPC A61K 31/726; A61K 47/48246; A61K 47/4823
USPC 514/1.1; 514/42; 435/375; 536/29.1

(57) ABSTRACT

The present invention provides a compound comprising a helminth-derived glycan and/or glycoconjugate thereof (e.g., a compound comprising a Lewis^{x} antigen (e.g., LNFPIII), a non-Lewis^{x} antigen (e.g., LNnT, LDN, and LDN derivatives), or a mixture of Lewis^{x} and non-Lewis^{x} antigens (e.g., SEA)), useful as a therapeutic compound for treating or preventing diseases associated with fat accumulation in the liver. The compounds of the invention are useful for treating or preventing the development of a fatty liver disease in a subject that has the disease or is at risk of developing the disease, and inhibiting lipogenesis in hepatocytes. The invention also provides methods of regulating the Erk-c-fos/AP-1-FXRα signalling pathway by administering a compound comprising a helminth-derived glycan and/or glycoconjugate thereof to a subject with a fatty liver disease, or contacting a hepatocyte with a compound comprising a helminth-derived glycan and/or glycoconjugate thereof.

### RELATED APPLICATIONS

This application claim priority to U.S. Provisional Application No. 61/538,629, entitled “Methods of Treating Fatty Liver Disease with Helminth-Derived Glycan-Containing Compounds”, filed Sep. 23, 2011, the entire contents of which are incorporated herein by this reference.

### BACKGROUND OF THE INVENTION

Diseases characterized by fat accumulation in the liver are becoming increasingly prevalent in adults and children of industrialized countries due, in large part, to unhealthy eating habits and obesity. Alcoholic fatty liver disease (AFLD), which results from chronic excessive alcohol intake, essentially follows a pathological course involving steatosis, steatohepatitis (ASH), inflammation, cirrhosis, and, in some cases, hepatocellular carcinoma. Non-alcoholic fatty liver disease (NAFLD), considered the most common liver disease, is a term that encompasses a series of hepatic pathologies similar to those of AFLD that range in severity from hepatic steatosis (accumulation of fat in the liver), cirrhosis, to hepatocellular carcinoma. Accordingly, NAFLD exhibits the histological features of AFLD in patients without a history of alcohol abuse. All stages of AFLD and NAFLD have in common the accumulation of fat in hepatocytes (Reddy et al., Am J Physiol Gastrointest Liver Physiol 2006; 290:G852-8).

An estimated one third of the U.S. population is affected with NAFLD (Grattagliano et al., Can Fam Physician 2007; 53:857-863). Development of NAFLD appears to be gender-biased, with men being more likely to develop the disease compared to women, and at earlier ages (Browning et al. Hepatology 2004; 40:1387-95). NAFLD can progressively worsen from hepatic steatosis to non-alcoholic steatohepatitis (NASH), which is characterized by the development of liver injury as evidenced by hepatocyte injury, infiltration of inflammatory cells, or fibrosis. In turn, NASH can progress into liver cirrhosis, which is associated with the replacement of hepatocytes with scar tissue, and at more advanced stages, hepatocellular carcinoma. NAFLD is recognized as an important and common cause of cirrhosis and liver failure (Wanless et al, Hepatology, 1990; 12:1106-10). NAFLD is considered to be part of the metabolic syndrome (MetS). Similar to those with MetS, >80% of individuals with NAFLD are overweight, with approximately 30% being obese (Grattagliano et al., 2007). Furthermore, those with NAFLD also often have concurrent hyperlipidemia, type 2 diabetes mellitus (T2DM), and are hypertensive.

Hepatic steatosis is characterized by increased liver accumulation of triglycerides, major sources of which include diet, de novo fatty acid synthesis, and adipose tissue (Cohen et al., Science 2011; 332:1519-23). This accumulation of triglycerides, in turn, can lead to insulin resistance (Macias-Rodriguez et al., Rev Invest Clin 2009; 61:161-72). According to the multiple-hit hypothesis, the transition from hepatic steatosis to NASH requires multiple hits (Day et al., Gastroenterology 1998; 114:842-5). Increased hepatic free fatty acid levels due to impaired insulin sensitivity can serve as the first hit, resulting in insulin resistance, steatosis, lipid peroxidation, and increased inflammatory cell accumulation and activation (Schuppan et al., Liver International 2010; 30:795-808). This, in turn, can lead to lipotoxicity and hepatocyte growth arrest or apoptosis (Id.; Feldstein et al. Gastroenterology 2003; 125:437-43). A number of inherited disorders are associated with hepatic steatosis, such as glycogen storage disease type 1a, citrin deficiency, and congenital generalized lipodystrophy (Hooper et al., J Lipid Res 2011; 52:593-617; Bandsma et al., Pediatr Res 2008; 63:702-7; Komatsu et al., J Hepatol 2008; 49:810-20). Furthermore, mutations in genes that function in triglyceride homeostasis also play a role in the development of hepatic steatosis (Cohen et al., supra).

There is currently a lack of established treatments for NAFLD, and most treatments hinge on managing associated conditions such as obesity, diabetes mellitus, and hyperlipidemia (Angulo et al., Sem Liver Dis. 2001; 21(1):81-8). Other therapies for NAFLD mainly target lifestyle habits, such as exercise and diet. Pharmacological interventions aimed at targeting weight loss and insulin resistance have limited efficacy (Mishra et al. Curr Drug Discov Technol 2007; 4:133-40). However, some studies have shown signs of steatosis reversal after weight loss, suggesting that the condition may be reversible and providing hopes of developing therapies that target pathways involved in NAFLD pathogenesis to reverse disease progression (Eriksson et al., Acta Med Scand. 1986; 220:83-88; Sheth et al., Ann Intern Med 1997; 126(2):137-45). The main therapy for AFLD involve abstinence from alcohol (Scaglioni et al., Dig Dis 2011; 29:202-10).

Given the current lack of effective treatments against the spectrum of disorders associated with increased fat accumulation in the liver, in conjunction with the estimated future increase in prevalence of obesity and insulin resistance worldwide, there is an urgent need to develop new methods of preventing and treating these diseases. Further exacerbating this problem is the lack of a clear understanding of mechanisms involved in the pathogenesis of these diseases. A better understanding of molecular pathways involved in, e.g., hepatic lipogenesis or the development of AFLD and NAFLD, will provide avenues for developing targeted drug-based therapies.

### SUMMARY OF THE INVENTION

The invention is based, at least in part, on the discovery that a compound comprising a helminth-derived glycan, e.g., LNFPIII, reduces hepatic inflammation and inhibits fat accumulation in hepatocytes and the liver.

In one aspect, the invention provides a method of treating a disease associated with fat accumulation in the liver, comprising administering to a mammal in need thereof a therapeutically effective amount of a compound comprising a helminth-derived glycan and/or glycoconjugate thereof.

In another aspect, the invention provides a method of preventing a disease associated with fat accumulation in the liver comprising administering to a mammal at risk of developing said disease a prophylactically effective amount of compound comprising a helminth-derived glycan and/or glycoconjugate thereof.

In certain embodiments, the disease to be treated or prevented according to the methods of the invention is FLD, NAFLD or AFLD. In certain preferred embodiments, the disease is hepatic steatosis, non-alcoholic steatohepatitis, or alcoholic steatohepatitis. In yet another embodiment, the disease is metabolic syndrome.

In certain embodiments, the compound used in the methods of the invention comprising a helminth-derived glycan and/or glycoconjugate thereof reduces triglyceride levels in the mammal. In related embodiments, the compound comprising a helminth-derived glycan and/or glycoconjugate thereof suppresses or inhibits lipogenesis in the liver of the mammal. In other related embodiments, the compound comprising a helminth-derived glycan and/or glycoconjugate thereof increases the production of FXRα and/or reduces the production of SREBP-1c in hepatocytes.

In certain embodiments of the methods of the invention, the helminth-derived glycan and/or glycoconjugate containing compound is administered parenterally, preferably intraperitoneally or intravenously. In another embodiment, the compound is administered orally.

In another aspect, the invention provides a method of reducing lipogenesis in a hepatocyte comprising contacting the hepatocyte with a sufficient amount of a compound comprising a helminth-derived glycan and/or glycoconjugate thereof.

In another aspect, the invention provides a method of increasing the production of FXRα in a cell (e.g., a hepatocyte), comprising contacting the cell with a compound comprising a helminth-derived glycan and/or glycoconjugate thereof. In a preferred embodiment, the FXRα is FXRα3/α4. In another preferred embodiment, the FXRα is FXRα1/α2.

In another aspect, the invention provides a method of increasing the production of a gene product that is transcriptionally induced by FXRα, comprising contacting a hepatocyte with a compound comprising a helminth-derived glycan and/or glycoconjugate thereof. In preferred embodiments, the gene product is SHP, OATP1, PLTP, and/or BSEP.

In another aspect, the invention provides a method of increasing activation of the Erk-c-fos/AP1-FXRα pathway in a hepatocyte, comprising contacting the hepatocyte with a compound comprising a helminth-derived glycan and/or glycoconjugate thereof. In a preferred embodiment, activation of the Erk-c-fos/AP1-FXRα pathway is accompanied by a reduction in lipogenesis.

In certain embodiments, the compound used in the methods of the invention comprises at least one, or at least two, helminth-derived glycan and/or glycoconjugates. In certain embodiments, the helminth-derived glycan comprises a Lewis^{x} antigen, a non-Lewis^{x} antigen or a combination thereof. In certain preferred embodiments, the compounds used in the methods of the invention comprise LNFPIII, LNnT, LDN, LDNF, SEA, or combinations thereof.

In other embodiments, the helminth-derived glycan and/or glycoconjugate thereof used in the methods of the invention is crosslinked to a carrier molecule. In certain preferred embodiments, two or more helminth-derived glycans and/or glycoconjugates, are conjugated to the carrier molecule. In preferred embodiments, the molecular weight of the crosslinked helminth-derived glycan and/or glycoconjugate and carrier molecule is preferably about 5,000 to 100,000 daltons, and more preferably about 10,000 to 40,000 daltons. In yet other preferred embodiments, the crosslinked conjugate has 2-200 helminth-derived glycan and/or glycoconjugate-containing oligosaccharide molecules per carrier molecule, more preferably, 10-100 helminth-derived glycan- and/or glycoconjugate-containing oligosaccharide molecules per carrier molecule, and even more preferably 20-50 helminth-derived glycans and/or glycoconjugate-containing oligosaccharide molecules per carrier molecule. In certain preferred embodiments, the carrier is a carbohydrate polymer (e.g., dextran), protein (e.g., HSA), or polyacrylamide.

In a related aspect, the invention also features a pharmaceutical composition comprising one or more of the helminth-derived glyan and/or glycoconjugates in an amount sufficient to treat a disease or disorder associated with fat accumulation in the liver. Also included in the invention are kits comprising the compositions of the invention.

Other features and advantages of the invention will be apparent from the following detailed description and the claims.

### BRIEF DESCRIPTION OF THE FIGURES

FIG. 1A depicts micrographs of liver sections from vehicle, LNFPIII, or SEA treated mice. Male C57BL/6J mice at 8-10 weeks of age were placed on a high-fat, high carbohydrate diet for the duration of the experiments. After 6 weeks on this diet, mice were injected two times per week with 25 μg dextran (vehicle), 25 μg LNFPIII-dextran (LNFPIII), or 25 μg SEA (in 0.9% NaCl) as indicated. Vehicle for SEA experiments was 0.9% NaCl. Mice were sacrificed at the 6^{th} week of treatment for tissue collection and histology.

FIG. 1B is a bar graph depicting levels of triglycerides in livers from vehicle or LNFPIII treated mice (left panel) or SEA (25 μg in 0.9% NaCl) treated mice (right panel). Results are presented as mean±SEM. *p<0.05.

FIG. 1C is a bar graph depicting relative mRNA expression of genes involved in inflammation, lipogenesis, and β-oxidation in livers of mice treated with vehicle or LNFPIII (n=5/group) as assessed with real-time q-PCR. Results are presented as mean±SEM. *p<0.05.

FIG. 1D is a bar graph depicting levels of aspartate aminotransferase (AST) and alanine aminotransferase (ALT) in circulation of vehicle or LNFPIII treated mice. Results are presented as mean±SEM. *p<0.05.

FIG. 1E is a bar graph depicting levels of AST and ALT in circulation of vehicle or SEA treated mice. Results are presented as mean±SEM. *p<0.05.

FIG. 2A is a bar graph showing the degree of lipogenesis (left panel) or β-oxidation (right panel) in primary hepatocytes treated with vehicle (20 μg/ml) or LNFPIII (20 μg/ml) for 24 hours in vitro. Results are presented as mean±SEM. *p<0.05.

FIG. 2B is a bar graph depicting the mRNA expression levels of the indicated genes in primary hepatocytes treated with vehicle or LNFPIII for 24 hours. LNFPIII increases FXRα and SHP expression, but downregulates lipogenic genes. Results are presented as mean±SEM. *p<0.05.

FIG. 2C is a schematic representation of the genomic structure of the FXRα promoter showing alternative promoter usage to drive expression of human FXRα1/α2 (promoter 1) and FXRα3/α4 (promoter 2).

FIG. 2D and FIG. 2E are bar graphs depicting mRNA expression levels of genes that function in the FXRα signaling pathway in livers of mice treated with vehicle or LNFPIII (FIG. 2D) (n=5/group) or vehicle (0.9% NaCl) and SEA (25 μg in 0.9% NaCl) (FIG. 2E) (n=5/group) as assessed by real-time q-PCR. Results are presented as mean±SEM. *p<0.05.

FIG. 2F is a bar graph depicting relative mRNA expression of genes involved in lipogenesis and β-oxidation in livers of FXRα^{-/-} mice treated with vehicle or LNFPIII (n=6/group), as assessed with real-time q-PCR.

FIG. 2G is a bar graph depicting lipogenesis and β-oxidation in primary hepatocytes isolated from wild type mice or FXRα^{-/-} mice treated with vehicle, LNFPIII, or SEA. Cells were treated for 24 hours. Results are presented as mean±SEM. *p<0.05. The ability of LNFPIII and SEA to inhibit lipogenesis and enhance β-oxidation in hepatocytes is dependent on FXRα.

FIG. 2H is a bar graph depicting hepatic triglyceride content in wild type and FXR^{-/-} mice with or without LNFPIII treatment (n=6/genotype). Results are presented as mean±SEM. *p<0.05 (LNFPIII versus vehicle control).

FIG. 2I is a bar graph depicting serum AST and ALT concentrations in wild type and FXR^{-/-} mice with or without LNFPIII treatment (n=6/genotype). Results are presented as mean±SEM. *p<0.05 (LNFPIII versus vehicle control).

FIG. 3A is an alignment of sequences of the 5′ proximal regulatory region of human, rat, and mouse FXRα downstream promoter (promoter 2). Putative binding sites for C/EBP (site 1) and AP-1 (sites 2 and 3) and mutated sequences for the reporter assays are indicated.

FIG. 3B is a bar graph depicting a luciferase reporter assay of HepG2 cells transfected with luciferase reporter constructs driven by a 2 kb fragment of the human FXRα promoter 1 or 0.13 kb of human FXRα promoter 2 with or without LNFPIII (20 μg/ml) or SEA (2 μg/ml) treatment. Results are presented as mean±SEM. *p<0.05.

FIG. 3C is a bar graph depicting mRNA levels of endogenous FXRα1/α2 and FXRα3/α4 genes in primary hepatocytes treated with or without LNFPIII (20 μg/ml) or SEA (2 μg/ml). Results are presented as mean±SEM. *p<0.05.

FIG. 3D (top panel) is a schematic diagram showing FXRα promoter 2 (p2-0.13 kb) or mutant (p2-0.13 kb-M) constructs. The transcriptional start site was designated as 1. Sequences of the two overlapping AP1 binding sites and the mutations introduced are shown. FIG. 3D (bottom panel) is a bar graph depicting a luciferase reporter assay using the indicated constructs in HepG2 cells treated with or without vehicle or LNFPIII in the presence or absence of the Erk inhibitor PD98059 (10 μM). Cells were pretreated with PD98059 for one hour prior to overnight LNFPIII treatment. Results are presented as mean±SEM. *p<0.05.

FIG. 3E is a bar graph depicting a luciferase reporter assay with the Site 1 mutant, Site 2 mutant, or Site 3 mutant luciferase reporter constructs in HepG2 cells treated with vehicle or SEA. The sites are defined in FIG. 3A. Results are presented as mean±SEM. *p<0.05. Induction of the FXRα promoter 2 is mediated by AP1 binding sequences in site 3. Mutations in sites 1 and 2 had no effect. Similar results were obtained with LNFPIII (data not shown).

FIG. 3F (top panel) is an immunoblot for levels of phosphor-Erk (p-Erk) and total Erk (t-Erk) in liver lysates prepared from vehicle or LNFPIII treated mice (n=5, showing representative 3 samples/treatment). Actin levels served as a loading control. FIG. 3F (bottom panel) is a bar graph depicting the densitometric analysis of the immunoblot from FIG. 3F (top panel). Results are presented as mean±SEM. *p<0.05.

FIG. 3G (left panel) is an immunoblot for levels of phospho-Erk (p-Erk) and total Erk (t-Erk) in primary hepatocytes treated with vehicle, LNFPIII, PD90859, or LNFPIII+PD98059. Cells were pretreated with PD98059 for 1 hour prior to overnight LNFPIII treatment. Actin levels served as a loading control. FIG. 3G (right panel) is a bar graph depicting the densitometric analysis of the immunoblot from FIG. 3G (left panel). Results are presented as mean±SEM.

FIG. 3H is an immunoblot for levels of p-Erk and t-Erk in primary hepatocytes treated with vehicle, SEA, or SEA+PD98059. Cells were pretreated with PD98059 for 1 hour prior to overnight SEA treatment. Actin levels served as a loading control.

FIG. 3I (left panel) is a bar graph depicting lipogenesis activity in WT and FXRα^{-/-} hepatocytes treated with vehicle, LNFPIII, PD98059, or LNFPIII+PD98059. Cells were pretreated with PD98059 for 1 hour prior to overnight LNFPIII treatment. Results are presented as mean±SEM. *p<0.05. The ability of LNFPIII to suppress lipogenesis in hepatocytes was dependent on Erk activity and FXRα. FIG. 3I (right panel) is a bar graph depicting β-oxidation in WT and FXRα^{-/-} hepatocytes treated with vehicle, LNFPIII, PD98059, or LNFPIII+PD98059. Cells were treated as in the lipogenesis assay. Results are presented as mean±SEM. *p<0.05. The ability of LNFPIII to increase β-oxidation in hepatocytes was dependent on Erk activity and FXRα.

FIG. 3J is a bar graph depicting lipogenesis activity of hepatocytes treated with vehicle, SEA, or SEA+PD98059. Cells were pretreated with PD98059 for 1 hour prior to overnight SEA treatment. Results are presented as mean±SEM. *p<0.05. The ability of SEA to suppress lipogenesis in hepatocytes was dependent on Erk activity.

FIG. 4A is a bar graph depicting lipogenesis activity of hepatocytes treated with vehicle (first lane), 5 μg LNnT (second lane), or 20 μg LNnT (third lane). Results are presented as mean±SEM. *p<0.05.

FIG. 4B (upper panel) is a schematic representing the genomic structure of the FXRα promoter showing alternative promoter usage to drive expression of human FXRα1/α2 and FXRα3/α4. FIG. 4B (lower panel) is a bar graph depicting a luciferase reporter assay of HepG2 cells transfected with luciferase reporter constructs driven the human FXRα1/α2 promoter or FXRα3/α4 promoter with or without LNnT. Results are presented as mean±SEM. *p<0.05.

FIG. 5 is a schematic diagram of the Erk-c-fos/AP1-FXRα axis signaling pathway.

### DETAILED DESCRIPTION OF THE INVENTION

Prior to the present invention, it was known that compounds comprising a Lewis antigen (e.g., LNFPIII) can regulate the production of cytokines (e.g., IL-10, IL-4, and IL-5) by immune cells that skew the development of Th1 or Th2 responses, which are known to be important for the development and progression of many disease states, such as autoimmune disorders, cancer and infectious diseases. For example, stimulatory forms of Lewis antigen compounds have been shown to elicit antigen-specific immune responses (IgG responses), whereas inhibitory forms have been shown to inhibit allergic responses (IgE responses) (e.g., see, U.S. Pat. Nos. 6,540,999, 6,841,543, and 7,799,755. However, the roles of Lewis antigen containing compounds (e.g., LNFPIII) in other clinical contexts and the signalling pathways targeted by these compounds were essentially unknown.

Accordingly, the present invention is based, at least in part, on the unexpected finding that a compound comprising a helminth-derived glycan or glycoconjugate thereof (e.g., a compound comprising a Lewis^{x} antigen (e.g., LNFPIII), a non-Lewis^{x} antigen (e.g., LNnT, LDN, derivatives thereof), or a mixture of Lewis^{x} and non-Lewis^{x} antigens (e.g., SEA)), can activate Erk, followed by activation of the AP-1 transcriptional complex, induction of FXRα and FXRα target gene expression, and FXRα-mediated inhibition of lipogenesis in hepatocytes. The reduction in lipogenic gene expression is accompanied by reduced triglyceride levels and increased fatty acid β-oxidation in hepatocytes and the liver.

Thus, the present invention provides methods of treating or preventing diseases characterized by increased inflammation and fat accumulation in the liver with a compound comprising a helminth-derived glycan and/or glycoconjugate thereof (e.g., a compound comprising Lewis antigen, such as LNFPIII and SEA). The methods of the invention are useful for treating or preventing the development of fatty liver disease (FLD) in a subject that has or is at risk of developing the disease, and for inhibiting lipogenesis in hepatocytes. The invention also provides methods of regulating the Erk-c-fos/AP-1-FXRα signalling pathway by administering a compound comprising a helminth-derived glycan and/or glycoconjugate thereof to a subject with a fatty liver disease, or contacting a hepatocyte with a compound comprising a helminth-derived glycan and/or glycoconjugate thereof.

Erk belongs to a subfamily of mitogen-activated protein kinases (MAPK), the activation of which by successive phosphorylation is secondary to extracellular stimuli binding to their receptors on the cell surface. Upon activation, Erk phosphorylates and activates various cytoplasmic and nuclear proteins, and mediates multiple biological responses including those that control cell growth and differentiation. In the context of the present invention, treatment of hepatocytes or subjects with a compound comprising a helminth-derived glycan and/or glycoconjugate thereof (e.g., a compound comprising Lewis antigen such as LNFPIII and SEA) increases Erk activity.

The AP-1 complex is formed through the dimerization of c-fos, a bZIP type transcription factor, and c-jun. The AP-1 complex is activated by the MAPK signalling cascade in response to extracellular signals. In the context of the present invention, treatment of hepatocytes or subjects with a compound comprising a helminth-derived glycan and/or glycoconjugate thereof (e.g., a compound comprising Lewis antigen such as LNFPIII and SEA) increases AP-1 activity.

FXR, or the bile acid receptor, is a nuclear receptor that is highly expressed in the liver and intestine. FXR is one of the key transcription factors responsible for regulating bile acid homeostasis (Forman et al., Cell 1995; 81:687-93). Upon activation, FXR translocates to the cell nucleus, dimerizes with a binding partner, and binds to hormone response elements on DNA. Target genes of FXR can be direct or indirect. Direct regulation of target genes can occur through the direct binding of hormone response elements on DNA. Indirect regulation of transcriptional targets can occur through an intermediate. For example, FXR can induce the expression of small heterodimer partner (SHP) protein, which can then inhibit the transcription of target genes. Mechanistically, the FXR-mediated induction of SHP has been shown to decrease the expression of the master lipogenic regulator SREBP-1c (Watanabe et al., J Clin Invest 2004; 113:1408-18). Some examples of FXRα target genes include SHP, organic anion transport protein-1 (OATP1), phospholipid transfer protein (PLTP), and bile salt excretory pump (BSEP). In the context of the present invention, treatment of hepatocytes or subjects with a compound comprising a helminth-derived glycan and/or glycoconjugate thereof, e.g., LNFPIII and SEA, increases FXRα, specifically FXRα3/α4 production. Treatment of hepatocytes or subjects with a SEA increases both FXRα1/α2 and FXRα3/α4 production.

Lipogenic genes that are downregulated in response to treatment with a compound comprising a helminth-derived glycan and/or glycoconjugate thereof (e.g., a compound comprising Lewis antigen such as LNFPIII and SEA) include SREBP-1c, and its downstream transcriptional target genes [e.g., fatty acid synthase (FAS), acetyl-Co A carboxylase-1 (ACC1), acetyl-Co A carboxylase-2 (ACC2), stearoyl CoA desaturase 1 (SCD1)]. SREBP-1c is a member of sterol regulatory element binding proteins (SREBPs), which are transcription factors belonging to the bHLH-Zip family (Yokoyama et al., Cell 1993; 75:187-97). These transcription factors bind to sterol regulatory elements (SRE), which are present in genes encoding enzymes that participate in cholesterol or fatty acid biosynthesis (Rawson et al., MCB 2003; 4:631-40).

The various compositions and methods of the invention are described in detail below. Although particular compositions and methods are exemplified herein, it is understood that any of a number of alternative compositions and methods are applicable and suitable for use in practicing the invention.

### DEFINITIONS

Unless otherwise indicated, all terms used herein have the same meaning as they would to one skilled in the art, and the practice of the present invention will employ conventional techniques of microbiology and recombinant DNA technology, which are within the knowledge of those of skill in the art. The practice of the present invention will employ, unless otherwise indicated, conventional methods of virology, microbiology, molecular biology and recombinant DNA techniques within the skill of the art. Such techniques are explained fully in the literature. See, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijssen, ed.); Fundamental Virology, 2nd Edition, vol. I & II (B. N. Fields and D. M. Knipe, eds.)

As used herein, the term “Lewis antigen” is intended to include carbohydrates having as a core sequence either the lacto type I structure {Gal(β1-3)GlcNac} or the lacto type II structure {Gal(β1-4)GlcNac}, substituted with one or more fucosyl residues. The Lewis antigen may comprise a single substituted core sequence or a repetitive series of substituted core sequences. Moreover, the core sequence may be present within a larger sugar. Accordingly, a Lewis antigen-containing oligosaccharide can be, for example, a trisaccharide, a tetrasaccharide, a pentasaccharide, and so on. Types of Lewis antigens include Lewis^{x}, Lewis^{y}, Lewis^{a}, and Lewis^{b} oligosaccharides and derivatives thereof. Synthetic structural homologues of these carbohydrates that retain the immunomodulatory capacity described herein are also intended to be encompassed by the term “Lewis antigen”.

As used herein, the term “Lewis^{x} oligosaccharide” refers to a lacto type II carbohydrate comprising the structure: {Gal(β1-4)[Fuc(α1-3)]GlcNac}.

As used herein, the term “Lewis^{y} oligosaccharide” refers to a lacto type II carbohydrate comprising the structure: {Fuc(α1-2)Gal(β1-4)[Fuc(α1-3)]GlcNac}.

As used herein, the term “Lewis^{a} oligosaccharide” refers to a lacto type I carbohydrate comprising the structure: {Gal(β1-3)[Fuc(α1-4)]GlcNac}.

As used herein, the term “Lewis^{b} oligosaccharide” refers to a lacto type I carbohydrate comprising the structure: {Fuc(α1-2)Gal(β1-3)[Fuc(α1-4)]GlcNac}.

As used herein, a “derivative” of a Lewis oligosaccharide refers to a Lewis oligosaccharide having one or more additional substituent groups. Examples of derivatives include terminally sialylated forms of Lewis oligosaccharides (e.g., sialyl-Lewis^{x}, sialyl-Lewis^{y}, sialyl-Lewis^{a}, sialyl-Lewis^{b}), sulfated forms of Lewis oligosaccharides, and sulfo-sialylated forms of Lewis oligosaccharides.

As used herein, the term “SEA” refers to schistosome egg antigen or soluble egg antigen, which comprises Lewis^{x} and non-Lewis^{x} antigens. “SEA-derived glycans” refer to the various different glycan species present in SEA, such as, but not limited to, LNFPIII, LNnT, LDN, and LDNF.

As used herein, the term “LN” refers to a sugar with the structure: {Gal(β1-4)GlcNAc}.

As used herein, the term “LNFPIII” refers to a compound with the structure: {Gal(β1-4)[Fuc(α1-3)]GlcNAc(β1-3)Gal(β1-4)Glc} and comprises the Lewis^{x} antigen.

As used herein, the term “LNnT” (lacto-N-neotetraose) refers to a polylactosamine sugar having the core structure: {Gal(β1-4)GlcNAc(β1-3)Gal(β1-4)Glc}. LNnT is a non-fucosylated homologue of LNFPIII.

As used herein, the term “LDN” (LacdiNAc) refers to a sugar with the structure: {GalNAc(β1-4)GlcNAc}. Fucosylated LDN, herein referred to as “LDNF” has the structure: {GalNac(β1-4)(Fucα1-3)GlcNAcβ1}. Other fucosylated derivatives of LDN include, but are not limited to, LDN-DF {GalNAc(β1-4)[Fuc(α1-2)Fuc(α1-3)]GlcNAcβ1}, F-LDN {Fuc(α1-3)GalNAc(β1-4)GlcNAcβ1}, F-LDN-F {Fuc(α1-3)GalNAc(β1-4)[Fuc(α1-3)]GlcNAcβ1}, and DF-LDN-DF {Fuc(α1-2)Fuc(α1-3)GalNAc(β1-4)[Fuc(α1-2)Fuc(α1-3)]GlcNAcβ1}(Peterson et al., Int J Parasitology 2009; 39:1331-44; Hokke et al., Exp Parasitology 2007; 117:275-83). Both LDN and its fucosylated derivatives are considered within the scope of the invention.

As used herein, the term “helminth-derived glycan” refers to the glycan species present in eukaryotic parasitic worms classified as helminths, such as those reviewed in Jonhston et al., Parasitology 2009; 136:125-47 and Die and Cummings, Glycobiology 2010; 20:2-12 (for example, but not limited to, worms of the Schistosoma genus, such as S. mansoni; Fasciola genus, such as Fasciola hepatica; Echinococcus genus, such as Echinococcus multilocularis). Such glycans include compounds comprising a Lewis^{x} antigen (e.g., LNFPIII), a non-Lewis^{x} antigen (e.g., LNnT and LDN (and LDN derivatives)), or a mixture of Lewis^{x} and non-Lewis^{x} antigens (e.g., SEA). “Glycoconjugate” as used herein, refers to glycan molecules conjugated to carrier molecules, e.g., helminth-derived glycans conjugated to, e.g., lipids (e.g., glycolipids, phospholipids), proteins.

As used herein, the term “multivalent helminth-derived glycan or glycoconjugate thereof” is intended to refer to a synthesized construct comprising multiple copies of one or more helminth-derived glycan-containing oligosaccharides, such as a form in which multiple LNFPIII-containing oligosaccharides are conjugated to a carrier molecule. “Multivalent helminth-derived glycan and/or glycoconjugate thereof” can also refer to a form in which multiple different helminth-derived glycan and/or glycoconjugate species are conjugated to a carrier molecule. In a non-limiting example, a carrier molecule can be conjugated with two or more of LNFPIII, LNnT, LDN, and LDNF.

As used herein, the term “fatty liver disease” (FLD) is intended to encompass alcoholic fatty liver disease (AFLD) and non-alcoholic fatty liver disease (NAFLD).

As used herein, the term “alcoholic fatty liver disease” is intended to encompass alcoholic hepatic steatosis (ASH) and alcoholic steatohepatitis. AFLD is caused by fat accumulation in liver cells resulting from a history of alcohol consumption (Reddy et al., supra).

As used herein, the term “non-alcoholic fatty liver disease” is intended to refer to the spectrum of disorders resulting from an accumulation of fat in liver cells in individuals with no history of excessive alcohol consumption. In the mildest form, NAFLD refers to hepatic steatosis. The term NAFLD is also intended to encompass the more severe and advanced form non-alcoholic steatohepatitis (NASH), cirrhosis, hepatocellular carcinoma, and virus-induced (e.g., HIV, hepatitis) fatty liver disease.

As used herein, the term “subject” or “individual” or “patient” is intended to include human and nonhuman animals who have or are at risk of developing FLD. The term “mammals” of the invention includes all vertebrates, e.g., humans, nonhuman primates, sheep, dogs, cats, horses, cows, rodents and transgenic non-human animals. The terms “subject,” “individual,” and “mammals” are intended to be interchangeable.

As used herein, the term “contacting” (i.e., contacting a cell with a compound) is intended to include incubating the compound and the cell together in vitro (e.g., adding the compound to cells in culture) and administering the compound to a subject such that the compound and cells of the subject are contacted in vivo.

The term “treating” as used herein, refers to the application or administration of a composition comprising a helminth-derived glycan, a glycoconjugate-containing helminth-derived glycan, or a combination thereof to a subject to reduce, alleviate, inhibit the progress of, or eliminate, either partially or completely, one or more conditions or symptoms of the FLD (therapy). The term “treatment” as used herein, refers to the act of treating.

The terms “prevention”, “prevent” or “preventing” as used herein refers to the application or administration of a composition comprising a helminth-derived glycan, a glycoconjugate-containing helminth-derived glycan, or combination thereof to a subject to inhibit, avert or obviate the onset or progression of a disease (prophylaxis).

As used herein, the terms “sufficient amount” or “effective amount” are used interchangeably to refer to an amount necessary achieve a desired result. For example, a sufficient or effective amount of a helminth-derived glycan, glycoconjugate-containing compound (e.g., SEA, LNFPIII, LnNT, LDN, or LDNF), or a combination thereof results in one or more of the following: a decrease in the expression of one or more genes involved in lipogenesis, an increase in Erk phosphorylation, an increase in c-fos/AP1 transcriptional activity, an increase in transcription of the FXRα gene, and/or an increase in the production of transcriptional targets of the FXRα gene in an hepatocyte in vitro or in the liver in vivo, compared to a vehicle or control. An effective or sufficient amount of a helminth-derived glycan, glycoconjugate-containing compound (e.g., SEA, LNFPIII, LnNT, LDN, or LDNF), or combination thereof, also refers to an amount which is effective, either alone or in combination with a pharmaceutically acceptable carrier, for treating or preventing one or more symptoms or conditions associated with FLD. These effects can be ascertained using routine diagnostic techniques known in the art and may manifest as a prolongation of the survival of a subject, reduction in symptoms experienced by the subject, or the prevention of onset of disease in a subject.

Similarly, the term “therapeutically effective amount” refers to an amount effective, at dosages and for periods of time necessary, to achieve the desired therapeutic result. A therapeutically effective amount of a compound comprising a helminth-derived glycan and/or glycoconjugate thereof (e.g., a compound comprising Lewis antigen such as LNFPIII and SEA) may vary according to factors such as the disease state, age, sex, and weight of the individual and the ability of the vector to elicit a desired response in the individual. A therapeutically effective amount is also one in which any toxic or detrimental effect is outweighed by the therapeutically beneficial effects. For example, a therapeutically effective amount is an amount effective, at dosages and for periods of time necessary, to achieve a reduction in liver fat content to thereby influence the therapeutic course of a particular disease state. As used herein, the term “prophylactically effective amount” refers to an amount effective, at dosages and for periods of time necessary, to achieve the desired prophylactic result, for example, reducing or inhibiting the accumulation of fat in the liver for prophylactic purposes. Typically, since a prophylactic dose is used in subjects prior to or at an earlier stage of disease, the prophylactically effective amount will be less than the therapeutically effective amount.

Various aspects of the invention are described in further detail in the following subsections.

### I. Compounds for Treating or Preventing FLD

Compounds which may be used in the methods of the invention inhibit the accumulation of fat in the liver by suppressing lipogenesis in hepatocytes and/or protecting the liver against hepatic steatosis.

In one embodiment, the compound used in the methods of the invention comprises a helminth-derived glycan and/or glycoconjugate thereof, e.g., a compound comprising a Lewis antigen. The Lewis antigens can be, for example, Lewis^{x}, Lewis^{y}, Lewis^{a} or Lewis^{b} oligosaccharides, or derivatives thereof. In certain embodiments for the treatment of hepatocytes, the Lewis antigen is preferably Lewis^{x}. The Lewis antigen can also be present within a larger carbohydrate structure. For example, the carbohydrate portion of the compound can be lacto-N-fucopentaose III (LNFPIII), which has the structure: {Gal(β1-4)[Fuc(α1-3)]GlcNAc(β1-3)Gal(β1-4)Glc} and comprises the Lewis^{x} oligosaccharide.

Non-Lewis glycans include, but are not limited to, lacdiNAc (LDN), which has the structure: {GalNAc(β1-4)GlcNAc}; and fucosylated LacdiNAc (LDNF), which has the structure: {GalNac(β1-4)(Fucα1-3)GlcNAcβ1}. In another embodiment, the compound can comprise SEA-derived glycans. In another embodiment, the helminth-derived glycan is lacto-N-neotetraose (LNnT), which has the core structure: {Gal(β1-4)GlcNAc(β1-3)Gal(β1-4)Glc}.

Other related carbohydrates comprising Lewis or non-Lewis antigens that are suitable for use as a compound of the invention will be apparent to those skilled in the art. In a preferred embodiment, the helminth-derived glycan is LNFPIII. In another embodiment, compounds used in the methods of the invention can be administered in combination, either simultaneously or separately.

In certain embodiments, a compound comprising a helminth-derived glycan and/or glycoconjugate thereof typically used in the methods of the invention is one in which the carbohydrate structure is present in a multivalent, crosslinked form. In a preferred embodiment, a compound comprising a helminth-derived glycan and/or glycoconjugate thereof is a conjugate of a carrier molecule and multiple carbohydrate molecules expressing a helminth-derived glycan and/or glycoconjugate thereof. For example, carbohydrate molecules can be conjugated to a protein carrier, such as a conjugate of human serum albumin (HSA) and Lewis^{x} oligosaccharides (referred to herein as HSA-Lewis^{x}). When a sugar-carrier protein conjugate is to be administered to a subject, the carrier protein should be selected such that an immunological reaction to the carrier protein is not stimulated in the subject (e.g., a human carrier protein should be used with a human subject, etc.).

Alternative to a carrier protein, carbohydrate molecules expressing Lewis or non-Lewis antigens can be conjugated to other carrier molecules, for example carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparing such formulations will be apparent to those skilled in the art. The materials can also be obtained commercially from, for example, Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art as described in, for example, U.S. Pat. No. 4,522,811.

Other preferred carriers include polymers, such as carbohydrate or polysaccharide polymers. A preferred carbohydrate polymer is dextran. Typically, carbohydrates or polysaccharides that are useful as a carrier molecule have a molecular weight of about 5,000 to 100,000 daltons, preferably between about 8,000 to 80,000 daltons, more preferably about 10,000 to 50,000, or 10,000 to 40,000 daltons.

With respect to the density of sugars conjugated to the carrier, the sugar molecules preferably comprise at least 10% of the conjugate by weight, more preferably at least 15% of the conjugate by weight, even more preferably at least 20% of the conjugate by weight and even more preferably at least 25% of the conjugate by weight and even more preferably at least 25% of the conjugate by weight or at least 30% of the conjugate by weight or at least 35% of the conjugate by weight or at least 40% of the conjugate by weight or at least 45% of the conjugate by weight. In certain embodiments, the sugar molecules comprise about 10-25% of the conjugate by weight, about 15-25% of the conjugate by weight or about 20-25% of the conjugate by weight or about 30-35% by weight or about 35-40% by weight or about 40-45% by weight. In some embodiments, the conjugates comprise 10-11, 12-13, 14-15, 6-17, 18-19, or 20 or more sugars/conjugate. Even more preferably, the compound comprising a carbohydrate expressing a Lewis antigen is a conjugate containing 25, 30, 25, 40, 45, 50 or more sugars/conjugate. In a preferred embodiment, the compound comprising a carbohydrate expressing a Lewis antigen is a conjugate of multiple carbohydrate molecules expressing a Lewis antigen and the carrier polyacrylamide. More preferably, the polyacrylamide conjugates comprise 25 to 30 (or more) sugars/conjugate, wherein the average molecular weight of the conjugate is approximately 30 kD. Alternatively, or in combination with Lewis antigen(s), compounds can comprise a carbohydrate expressing a non-Lewis antigen.

In addition to conjugates comprising Lewis and/or non-Lewis antigen-containing sugars described above, other compounds comprising a Lewis and/or non-Lewis antigen include isolated proteins that naturally contain Lewis and/or non-Lewis antigens in a form suitable for suppressing lipogenesis and preventing or treating FLD. One example of such a protein is schistosome egg antigen (SEA), which expresses both Lewis^{x} and non-Lewis^{x} oligosaccharides. SEA can be isolated from various species. The genus Schistosoma contains 21 species of worm that have various life cycles. Of these, three main species are considered to cause schistosomiasis in humans—S. mansoni, S. japonicum, and S. haematobium. Eggs from all three species have been shown to induce a Th2 phenotype in humans. Antigens from all three species contain glycans that mimic host glycans. These include, for example, Lewis^{X}, pseudo Lewis^{Y}, LDN, LDNF, high mannose-type, and LN glycans. Similar structural glycans are found in eggs of most schistosome species. S. mansoni and S. japonicum are hepatic parasites, suggesting that they deposit eggs in the liver and hepatic circulation. Other proteins that have been reported to express Lewis antigens include tumor-associated antigens (see e.g., Pauli et al., Trends in Glycoscience and Glycotechnology 1992; 4:405-14; Hakomori, Adv Cancer Res 1989; 52:257-331) and HIV gp120 (Adachi et al., J Exp Med 1988; 167:323-31).

Compounds for use in the methods of the invention can be purchased commercially or can be purified or synthesized by standard methods. For example, conjugates of Lewis antigen-containing sugars and a carrier protein (e.g., HSA) are available from Accurate Chemicals, Westbury, N.Y. Conjugates of Lewis antigen-containing sugars and polyacrylamide are available from GlycoTech, Rockville, Md. LNFPIII is available from Sigma-Aldrich (USA). Schistosome egg antigen (SEA) can be purified from Schistosoma mansoni eggs as described in Ham et al. (1984) J. Exp. Med. 159:1371-1387. Lewis antigen-containing sugars, or derivatives thereof, can be conjugated to a carrier molecule by standard methods, for example using a chemical cross-linking agent. A wide variety of bifunctional or polyfunctional cross-linking reagents, both homo- and heterofunctional, are known in the art and are commercially available (e.g., Pierce Chemical Co., Rockford, Ill.). More than one species of helminth-derived glycan and/or glycoconjugate thereof can be conjugated to a single carrier. In one non-limiting example, one or more of LNFPIII, LnNT, LDN, and LDNF can be conjugated to the same carrier molecule. Such conjugations can be carried out by conjugating a mixture of heliminth-derived glycans, rather than a single helminth-derived glycan, to a carrier molecule.

Multivalent forms of helminth-derived glycans and/or glycoconjugates thereof can be generated using standard methods. For example, the oligosaccharide portion of the helminth-derived glycan and/or glycoconjugate thereof is bound to a multivalent carrier using techniques known in the art so as to produce a conjugate in which more than one individual molecule of the oligosaccharide is covalently attached to the multivalent carrier. The multivalent carrier is sufficiently large to provide a multivalent molecule leaving from between 2-1,000 (i.e. p=an integer of 2-1,000), preferably 2-200, preferably 2-100, more preferably, 10-100, more preferably 2-50, more preferably 10-50, even more preferably 20-50 molecules of the oligosachamide portion bound to the multivalent carrier. In certain preferred embodiments, 13, 25, 35, 45, 50, 100 or 200 helminth-derived glycan- and/or glycoconjugate-containing molecules, e.g., LNFPIII, are bound to the multivalent carrier.

Suitable multivalent carriers include compounds with multiple binding sites capable of forming a bond with a terminal linking group which is capable of binding to the reducing end saccharide, or with multiple binding sites capable of forming a bond to the C₁ glycosidic oxygen of a glucose or N-acetylglucosamine residue. Examples include, but are not limited to, a polyol, a polysaccharide, polylysine avidin, polyacrylamide, a carbohydrate (e.g., dextran), lipids, lipid emulsions, liposomes, a dendrimer, a protein (e.g., human serum albumin (HSA), bovine serum albumin (BSA)), or a cyclodextran.

The chemistry necessary to create the multivalent molecule and to link the oligosaccharide to the multivalent carrier are well known in the field of linking chemistry. Non-limiting examples of linkage chemistry in accordance with the present invention include those described in U.S. Pat. No. 5,736,533, Stowell et al. (Stowell et al. Advances in Carbohydrate Chemistry and Biochemistry 1980; 37:225) and Smith et al. (Smith et al. (1978) Complex Carbohydrates part C, Methods in Enzymology, volume L, Ed. by V. Ginsburg, pg. 169), each of which is hereby incorporated by reference herein.

For example, a bond between the reducing end saccharide and the carrier molecule can be formed by reacting an aldehyde or carboxylic acid at C1 of the reducing end saccharide or any aldehyde or carboxylic acid group introduced onto the reducing end saccharide by oxidation, with the carrier molecule to form a suitable bond such as —NH—, —N(R′)_where R′ is C1-20 alkyl, a hydroxyalkylamine, an amide, an ester, a thioester, a thioamide. The bond between the reducing end saccharide and the carrier molecule can also be formed by reacting the C1 hydroxyl group, in the pyranose form, with an acylating agent and a molecular halide, followed by reaction with a nucleophile to form a suitable bond such as —NH—, —N(R′)— where R′ is C1-20 alkyl (as described by Stowell et al., supra). The oligosaccharide portion can be bound to the multivalent carrier via the free anomeric carbon of the reducing end saccharide. Alternatively, the reducing end saccharide can be bound via a phenethylamine-isothyocyanate derivative as described by Smith et al., supra. A wide variety of other bifunctional and polyfunctional cross-linking agents that can be used to form multivalent conjugates are known in the art and readily available (e.g., Pierce Chemical Co., Rockford, Ill.).

### II. Assays

The effectiveness of any Lewis and/or non-Lewis antigen containing compound in the methods of the present invention can be readily determined by methods known in the art, which measure one more characteristics of lipogenesis.

For example, in vitro assays may be used in which a hepatocyte is contacted with a compound comprising a Lewis and/or non-Lewis antigen and then tested to determine whether the compound inhibits lipogenesis. Inhibition of lipogenesis, as used herein, refers to a reduction in the concentration of triglycerides, and/or a reduction in lipogenic gene expression and activity upon administration of a compound or composition comprising a helminth-derived glycan and/or glycoconjugate thereof (e.g., a compound comprising Lewis antigen such as LNFPIII and SEA) compared to a vehicle or control treated sample or animal or a control from before treatment.

In certain embodiments, helminth-derived glycan- and/or glycoconjugate-containing compounds that are useful in the methods of the invention result in a reduction of the level of one or more fatty acids, preferably triglycerides. In preferred embodiments, the level of tryglycerides are reduced by at least 10%, at least 20%, at least 30%, at least 40%, at least 50%, at least 60%, at least 70%, at least 80%, or at least 90% or more. Triglyceride levels can be assayed enzymatically using commercially available kits from, e.g., Sigma-Aldrich (USA), and following the manufacturer's instructions. Preparation of liver tissue for analysis of triglyceride levels can be performed as disclosed in, e.g., Hong et al. (Hepatology 204; 40:933-941). Circulating AST and ALT levels can be determined using commercially available kits (e.g., Sigma, USA; Bayer Diagnostics, USA) and following the manufacturer's instructions. Levels of lipid accumulation in cultured hepatocytes or liver tissue can be determined using art-recognized methods, e.g., Oil red 0 staining (e.g., Rector et al., Am J Physiol Gastrointest Liver Physiol 2008; 294:G619-G626).

In certain embodiments, helminth-derived glycan- and/or glycoconjugate-containing compounds that are useful in the methods of the invention reduce the expression of one or more lipogenic genes (e.g., SREBP-1c, FAS, ACC1, ACC2, SCD1) by at least 10%, at least 20%, at least 30%, at least 40%, at least 50%, at least 60%, at least 70%, at least 80%, at least 90% or more upon treatment with a Lewis antigen containing compound according to the methods of the invention. Methods for determining levels of mRNA or expressed proteins can be carried out with art-recognized methods including, but not limited to, e.g., Western blot, ELISA, dot blot, radioimmunoassay, and flow cytometry, Northern blot analysis, RT-PCR, and real-time qPCR.

In one embodiment, the effectiveness of a compound comprising a helminth-derived glycan and/or glycoconjugate thereof in modulating the Erk-c-fos/AP1-FXRα signalling axis can be readily determined by methods known in the art, for example, as described in the Examples. For example, cultured hepatocytes can be treated with vehicle or a helminth-derived glycan- and/or glycoconjugate-containing compound overnight, lysed, and the extracts processed for immunoblot and/or real-time quantitative PCR analysis. Activation of the pathway is evidenced by an increase in Erk activity (as assessed with phospho-ERK antibody), increased FXRα mRNA and downstream FXRα target gene expression (as assessed with, e.g., real-time qPCR), decreases in mRNA expression of SREBP1c and other lipogenic genes (as assessed with, e.g., real-time qPCR), decreased lipogenesis (as determined by, e.g., conversion of ¹⁴C-acetate to ¹⁴C-lipids extractable by chloroform/methanol (2:1) solution), and increased β-oxidation (as described in Reilly et al, Cell Metab 2010; 12:643-53). AP-1 activity can be determined using reporter assays (e.g., luciferase reporter assays) employing a suitable commercially available reporter (e.g., from QIAGEN, USA) or the FXRα3/α4 promoter as described in the Examples. AP-1 activity can also be determined by gel shift assays or immunprecipitation.

The efficacy of helminth-derived glycan- and/or glycoconjugate-containing compounds in the methods of the invention may also be tested in animal models of, for example, of NAFLD, AFLD and NASH. A compound (e.g., a compound comprising Lewis antigen such as LNFPIII and SEA) can be administered to test animals and the course of the disease in the test animals monitored by the standard methods for the particular model being used (e.g., histology, triglyceride levels, biomarker levels). Effectiveness of the compound is evidenced by amelioration or improvement of the disease condition, improvements in histology, biomarker levels (e.g., AST and ALT, which are hepatic enzymes that leak into general circulation upon hepatocyte injury), or triglyceride in animals treated with the compound as compared to untreated animals (or animals treated with a control compound or vehicle).

Non-limiting animal models of NAFLD and of NASH include, but are not limited to, the high fat diet induced obesity model described in the Examples; KK-Ay mice (CLEA Japan, Inc.) fed with a CMF diet (Oriental Yeast Co., Ltd, Japan) (U.S. 2011/0003757.); Long-Evans Tokushima fatty rats (Ota et al., Gastroenterology 2007; 32:282-93), SREBP-1c transgenic mice (Nakayama et al., Metabolism 2007; 56:470-75), L-SACC1 (CEA-related CAM) knockout mice (Lee et al., Gastroenterology 2008; 135:2084-95), LDL-R knockout+FXR knockout mice (Kong et al., J Pharmacol Exp Ther 2009; 328:116-22), and adipocyte 11β-HSD1 transgenic mice (Morton et al., Front Horm Res 2008; 36:146-64). Diet-based rodent NASH models include mice on an atherogenic diet (Matsuzawa et al., Hepatology 2007; 46:1392-403), rats treated with intragastric high polyunsaturated fatty acid (Baumgardner et al., Am J Physiol Gastrointest Liver Physiol 2008; 294:G27-38), mice on a methionine and choline-deficient (MCD) diet+high fat diet (HFD), rats on a MCD+trans-fats diethylnitrosamine (de Lima et al., J Hepatol 2008; 49:1055-56), and rats on a choline-deficient L-amino acid-defined diet (CDAA diet) (Hebbard et al., Nature Reviews Gastroenterology and Hepatology 2011; 8:35-44). Another NASH model is the obese Zucker rat (OZR), which is a spontaneous genetic obesity model that exhibits steatohepatitis and hyperphagia, hyperinsulinemia, and hyperlipidemia (Kitade et al., Hepatology 2006; 44:983-91).

Non-limiting examples of animal models for AFLD have also been described in, e.g., Gyamfi et al. (Exp Biol and Med 2010; 235:547-60); and the Lieber-DeCarli liquid diet and the Tsukamoto-French intragastric tubing feeding models (French. J Biomed Sci 2001; 8:20-7; Lieber et al., Hepatology 1989; 10:501-10).

Non-limiting animal models of hepatic fat accumulation in MetS can be tested in animal models of MetS, such as those described in, e.g., Panchal et al. (J Biomed Biotechnol 2011; 2011: 351982); high fat diet fed rats (Buettner et al., J Mol Endocrinol 2006; 36:485-501); high fructose, high fat-induced rats (Wada et al., Endocrinology 2010; 151:2040-9); high sucrose, high fat induced rats (Sato et al., Diabetes 2010; 59:2495-504); and Nile Grass rats (Noda et al., FASEB Journal 2010; 24:2443-53)

In addition, the toxicity and therapeutic efficacy of a helminth-derived glycan- and/or glycoconjugate-containing compound for use in the methods of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

### III. Pharmaceutical Compositions

Pharmaceutical compositions for use in the methods of the invention for the treatment of diseases associated with fat accumulation in the liver or hepatocytes typically comprise a compound comprising one or more helminth-derived glycans, glycoconjugates, or combinations thereof, preferably a compound comprising Lewis^{x} antigen such as LNFPIII and SEA, and a pharmaceutically acceptable carrier.

As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, transdermal or oral administration. In a preferred embodiment, the composition is formulated such that it is suitable for intravenous administration. A pharmaceutical composition of the invention can be formulated to be suitable for a particular route of administration. For example, in various embodiments, a pharmaceutical composition of the invention can be suitable for injection, inhalation or insufflation (either through the mouth or the nose), or for intranasal, mucosal, oral, buccal, parenteral, rectal, intramuscular, intravenous, intraperitoneal, and subcutaneous delivery.

Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the modulators can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Depending on the route of administration, the compound may be coated in a material to protect it from the action of enzymes, acids and other natural conditions which may inactivate the compound. For example, the compound can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluorophosphate (DEP) and trasylol. Liposomes include water-in-oil-in-water emulsions as well as conventional liposomes (Strejan et al., J Neuroimmunol 1984; 7:27). Dispersions can also be prepared in glycerol, liquid polyethylene glycols, and mixtures thereof and in oils. Under ordinary conditions of storage and use, these preparations may contain a preservative to prevent the growth of microorganisms.

The active compound in the composition preferably is formulated in the composition in a therapeutically effective amount. Dosage regimens may be adjusted to provide the optimum therapeutic response. A therapeutically effective amount is also one in which any toxic or detrimental effects of the compound are outweighed by the therapeutically beneficial effects.

In another embodiment, the active compound is formulated in the composition in a prophylactically effective amount. Typically, since a prophylactic dose is used in subjects prior to or at an earlier stage of disease, the prophylactically effective amount will be less than the therapeutically effective amount.

A non-limiting range for a therapeutically or prophylactically effective amounts of a compound of the invention is 0.01 nM-20 mM, preferably 0.1 nM-10 mM, and more preferably 1 μM-1 mM. Alternatively, the compound can be used twice a week in vivo in an amount between 1 μg to 100 mg/kg body weight, preferably 10 μg-200 mg/kg body weight, more preferably 20 μg-10 mg/kg body weight, and even more preferably 100 μg to 10 mg/kg body weight. The compound can be used in vitro in an amount between 0.01 μg/mL-100 μg/mL, more preferably 0.1 μg/mL-50 μg/mL, and even more preferably 2 μg/mL-20 μg/mL. It is to be noted that dosage values may vary with the severity of the condition to be alleviated and according to factors such as the disease state, age, sex, and weight of the subject.

It is to be further understood that for any particular subject, specific dosage regimens should be adjusted over time to provide the optimum therapeutic response according to the individual need and the professional judgment of the person administering or supervising the administration of the compositions, and that dosage ranges set forth herein are exemplary only and are not intended to limit the scope or practice of the claimed composition.

For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

A compound comprising a helminth-derived glycan and/or glycoconjugate thereof (e.g., a compound comprising Lewis antigen such as LNFPIII and SEA) of the invention can be formulated into a pharmaceutical composition wherein the compound is the only active compound therein. Alternatively, the pharmaceutical composition can contain additional active compounds. For example, more than one helminth-derived glycan and/or glycoconjugate thereof (e.g., LNFPIII, LNnT, LDN, or LDNF), can be used in combination. Moreover, more than one helminth-derived glycan and/or glycoconjugate thereof of the invention can be combined with one or more other agents that are used for the treatment of FLD, such as biguanides (e.g., metformin), thiazolidine derivatives (e.g., pioglitazone hydrochloride), α-glucosidase inhibitors (e.g., voglibose), insulin secretagogues (e.g., nateglinide), vitamins, eicosapentaenoic acid (EPA), betaine, N-acetylcysteine (NaC), fibrate drugs (e.g., bezafibrate), HMG-CoA reductase inhibitors (e.g., atorvastatin), probucol, ursodeoxycholic acid (UDCA), taurine, stronger neo-minophagen C, polyenephosphatidylcholine, angiotensin II receptor antagonists (e.g., losartan) or bofutsushosan (oriental herbal medicine), etc. When used in combination, drugs may be administered simultaneously or separately in succession or at desired time intervals. Formulations for simultaneous administration may be either mixed or separate form.

In certain embodiments, a pharmaceutical composition of the invention can be packaged with instructions for using the pharmaceutical composition for a particular purpose, such as to modulate an immune response, for use as an adjuvant, to modulate an allergic response or to modulate an autoimmune disease.

### IV. Methods of Treatment

For practicing the methods of the invention in vivo, a compound is administered to a subject in a pharmacologically acceptable carrier (as described supra) in an effective amount to achieve the desired effect, such as a reduction in lipogenesis in hepatocytes, a reduction in fat accumulation in the liver, and/or a reduction in the level of liver triglycerides. Any route of administration suitable for achieving the desired effect is contemplated by the invention. One preferred route of administration for the compound is parenteral. Another preferred route of administration is orally. Yet another preferred route of administration is intravenous. Application of the methods of the invention to the treatment of disease conditions may result in a decrease in the type or number of symptoms associated with the condition, either in the long term or short term (i.e., amelioration of the condition) or simply a transient beneficial effect to the subject.

In a related aspect, the invention provides a method for preventing or treating a disease or a disorder in a subject prophylactically or therapeutically. Administration of a compound prophylactically (i.e., the compound of the present invention) can occur prior to the manifestation of symptoms of an undesired disease or disorder, such that the disease or disorder is prevented or, alternatively, delayed in its progression. The prophylactic methods of the present invention can be carried out in a similar manner to therapeutic methods described herein.

Therapeutic effectiveness of the treatments can be demonstrated by improvements in liver histology (e.g., by liver biopsy), levels of fatty acids (e.g., triglycerides), and/or levels of biomarkers (e.g., circulating levels of AST and ALT) of subjects treated with a compound comprising a Lewis antigen. Changes in liver histology and/or levels of fatty acids and/or levels of biomarkers can be determined before and after intervention or in comparison with an untreated subject. Methods for carrying out these assays are described supra.

For each of the indicated treatments described herein, test subjects will exhibit a 5%, 10%, 20%, 30%, 50% or greater reduction, up to a 75-90%, or 95% or greater, reduction in one or more symptom(s) or biomarker levels caused by, or associated with, e.g., NAFLD, AFLD, and MetS, compared to levels prior to treatment or to placebo-treated or other suitable control subjects. In a specific embodiment, the level of one or more liver enzymes, preferably ALT or AST, is reduced by at least 10%, at least 20%, at least 30%, at least 40%, at least 50%, at least 60%, at least 70%, at least 80%, or at least 90% in a subject administered the treatments of the invention. In another embodiment, the level of one or more fatty acids, preferably triglycerides, in hepatocytes or liver tissue are reduced by at least 10%, at least 20%, at least 30%, at least 40%, at least 50%, at least 60%, at least 70%, at least 80%, or at least 90%.

Numerous disease conditions associated with an accumulation of fat in the liver have been identified and could benefit from administration of the compounds of the invention in the individual suffering from or at risk of developing the disease condition. Application of the compounds of the invention to such diseases is described in further detail below. For example, the fatty acid liver disorders that may benefit from the methods of the invention may be caused by a variety of factors, including but not limited to hepatitis, steatosis induced by viral or non-viral infectious agents, drug-induced steatosis (e.g., tamoxifen, uncoupling protein inhibitors, isoniazid, rifampicin, fibrates, peroxisome proliferator-activated receptor agonists), metabolic causes (e.g., obesity, polycystic ovary syndrome (PCOS), diabetes, insulin resistance, and metabolic disorder), alcohol-based causes (e.g., alcoholic fatty liver disease and alcoholic steatohepatitis), inborn errors of metabolism or genetic alterations (e.g., citrin deficiency, hemochromatosis, hyperferritinemia), inherited disorders (e.g., glycogen storage disease type 1a, citrin deficiency, and congenital generalized lipodystrophy), toxin-induced steatosis or steatohepatitis, celiac disease, lipodystrophy, bariatric surgery, and liver transplantation.

### A. NAFLD

The methods of the invention can be used to therapeutically or prophylactically treat or prevent the accumulation of fat in the liver. In particular, the methods are directed to treating or preventing NAFLD, a term that encompasses hepatic steatosis and NASH. Preferred compounds comprising a helminth-derived glycan and/or glycoconjugate thereof for treating or preventing NAFLD in a subject in need thereof are compounds comprising Lewis^{x} antigen, e.g., LNFPIII, and non-Lewis^{x} antigens, e.g., LNnT and SEA.

Several biomarkers are known to be useful for predicting, diagnosing, or staging NAFLD. Hepatic steatosis is defined as a hepatic TG level exceeding the 95^{th} percentile for lean, healthy individuals (i.e., >55 mg/g of liver) or as the presence of cytoplasmic TG droplets in more than 5% of hepatocytes (Cohen et al, supra). Those with NAFLD at risk for progression of hepatic steatosis to NASH can be identified based on unexplained increases to >2.5 times the upper limit of AST levels, ALT levels, or both, excessive body weight, T2DM, and unexplained hepatomegaly. Other markers include increased alkaline phosphatase, elevated ferritin levels, ALT concentrations exceeding AST levels, and unexplained increases in serum aminotransferase levels in overweight and obese individuals (Grattagliano et al., Can Fam Physician 2007; 53:857-86). Levels of triglycerides, AST, and ALT can be easily determined using the methods described infra or with commercially available kits. Another predictor that can be used for NASH is serum cytokeratin 18, the levels of which correlate with NASH activity (Wieckowska et al, Hepatology 2006; 44:27-33). Levels of cytokeratin 18 in serum of subjects at risk of developing NASH using commercially available ELISA kits (available from, e.g., Axxora LLC (San Diego, Calif.)) by taking serum from subjects at risk of developing NASH.

While determination of biomarker levels alone can be used to predict those at risk of developing NAFLD or diagnose those with NAFLD, these marker assays can be supplemented with histological assessments (e.g., liver biopsies) or non-invasive imaging modalities. Non-invasive imaging modalities known in the art for assessing NAFLD include, but are not limited to, magnetic resonance imaging (MRI), diffusion weighted MRI (Naganawa et al., Magn Reson Med Sci 2005; 4:175-86), and ultrasound elastography or MRI elastography (Friedrich-Rust et al., Gastroenterology 2008; 134:960-74; Talwalkar et al., Hepatology 2008; 47:332-42). Such non-invasive imaging modalities provide a useful and painless alternative for determining the presence of NAFLD and staging NAFLD progression.

Other non-invasive methods for determining mammals at risk for NAFLD and/or determining whether a mammal has a mild or severe form of NAFLD are disclosed in, e.g., U.S. Patent Publication No. 2009/0220986 (“the '986 publication”), which is herein incorporated by reference. For instance, the '986 publication discloses that a human with a low plasma level of DHEA-related compounds (e.g., <0.45 μg of DHEA or DHEA-S, the sulfated form of DHEA, per mL blood) can be classified as being at increased risk of having a severe form of NAFLD, such as severe NASH with more advanced liver fibrosis. Patients with a high plasma level of DHEA-related compounds (e.g., >0.45 μg of DHEA-S per mL blood) are highly unlikely to have severe NASH. Levels of DHEA related compounds in a sample can be readily determined using methods known in the art, including commercial radioimmunoassays and commercial ELISA kits (Diagnostic Systems Lab, Webster, Tex.). Samples, which can include, without limitation, blood, tissue (e.g., liver), or serum, can be isolated by any known method in the art. For instance, blood can be obtained using a needle or catheter, while a tissue sample can be obtained via biopsy.

Many genetic disorders are associated with severe hepatic steatosis, such as glycogen storage disease type 1a, citrin deficiency, congenital generalized lipodystrophy. Many genomic sequence variants associated with the full spectrum of NAFLD have been identified and can be used to determine those who are at risk of developing the disease (Cohen et al., supra). Such variants include mutations or sequence variants in genes such as ATGL, comparative gene identification-58 (CGI-58), hydroxyacyl-CoA transferases, VLDL, microsomal TG transfer protein, APOB, and PNPLA3 (Romeo et al., Nat Genet. 2008; 40:1461; Tanoli et al., J Lipid Res 2004; 45:941-7). In one embodiment, one at risk of developing NAFLD can be identified on the basis of the presence of a mutations in the PNPLA3 gene (e.g., Ile¹⁴⁸→Met¹⁴⁸). Such mutations in individuals can be readily identified DNA sequencing. Other identified genomic loci associated with hepatic steatosis include those coding for PPP1R3B, NCAN, GCKR, LYPLAL1, and APOC3 (Speliotes et al., PLoS Genetics 2011; 7:1-14; Cohen et al, supra). Thus, using routine techniques known in the art (i.e. DNA sequencing, RFLP analysis, genetic linkage studies), subjects can be identified who are at risk of developing NALFD and can thus be prophylactically treated with the compound of the present invention.

Another risk factor for developing NAFLD includes liver transplantation. Up to 70% of those who undergo liver transplantation for NASH or cryptogenic cirrhosis develop NALFD in the transplanted liver (Yalamanchili et al., Liver Transpl 2010; 16:431-9). Accordingly, individuals who undergo liver transplantations constitute a population at risk of developing NAFLD.

### B. Alcoholic Fatty Liver Disease (AFLD)

Alcoholic fatty liver disease shares the same pathologic features observed in NAFLD, except that, unlike individuals with NAFLD, those with AFLD have a history of alcohol abuse. One of the main therapies for AFLD involve abstinence from alcohol consumption, although this can be difficult to achieve. Preferred compounds comprising a helminth-derived glycan and/or glycoconjugate thereof for treating or preventing NAFLD in a subject in need thereof are compounds comprising Lewis^{x} antigen, e.g., LNFPIII, and non-Lewis^{x} antigens, e.g., LNnT and SEA.

Many factors can be assessed to determine susceptibility to ALD, such as patterns of alcohol intake, sex, age, and genetic polymorphisms (Becker et al., Hepatology 1996; 23:1025-9; Crabb et al., Proc Nutr Soc 2004; 63:49-63; Konishi et al., Exp Mol Pathol 2003; 74:183-9; Konishi et al., Alcohol Clin Exp Res 2004; 28:1145-52; Grove et al., Hepatology 1997; 26;143-6; Ladero et al., Scand J Gastroenterol 2005; 40:348-53; Savolainen et al., Alcohol Clin Exp Res 1996; 20:1340-5). Wan et al., Genet Test 1998; 2:79-83). Those with AFLD can be diagnosed using histological methods described in, for example, Tannapfel et al. (Virchows Arch 2011; 458:511-23).

Thus, in one embodiment, those at risk for developing AFLD can be readily determined by assessing the presence of genetic polymorphisms known to be associated with developing AFLD. Non-limiting examples of genetic polymorphisms associated with AFLD include, e.g., those identified in PPARγ2 (Rey et al., World J Gastroenterol 2010; 16:5830-7), mtDNA⁴⁹⁹⁷ deletion (Zhuang et al., CME, 2011 IEEE/ICME Intl Conference Jun. 16, 2011, pp. 127-31), and CYP2E1 (Maezawa et al., Am J Gastroenterol 1994; 89:561-5).

Individuals who have AFLD can be identified, as reviewed in Stickel et al. (Best Pract Res Clin Gastroenterol 2010; 24:683-93), on the basis of, e.g., various markers including elevated levels of neutrophils, serum bilirubin, and liver transaminases. Other factors that can be used to determine whether an individual has AFLD is a history of excessive alcohol consumption and exclusion of other similar etiologies. Yet other factors are pathophysiological, such as increased hepatic fat storage, increased hepatic uptake of gut-derived endotoxins that trigger activation of Kupffer cells, and ethanol-mediated hyperhomocysteinemia.

### C. Metabolic Syndrome

Metabolic syndrome is a collection of risk factors estimated to affect over 50 million Americans. There is currently a lack of well-accepted criteria for diagnosing metabolic syndrome, however, those that are affected exhibit abdominal obesity, atherogenic dislipidemia, elevated blood pressure, insulin resistance or glucose intolerance, prothrombotic state and a proinflammatory state. Individuals with metabolic syndrome are at risk for developing diseases that span fatty liver disease, peripheral vascular diseases, type 2 diabetes, and accelerated atherosclerosis.

In some embodiments of the invention, patients at risk of developing or who have NAFLD are affected by metabolic syndrome. Patients with metabolic syndrome, who are indicated for treatment with a compound comprising a Lewis and/or non-Lewis antigen according to the methods of the invention, can be identified via a number of established criteria. Throughout the years, many definitions have been set forth for what constitutes Met S. The main criteria currently used to diagnose Met S have been set forth by four groups: the World Health Organization (criteria established in 1999) (World Health Organization (WHO). Definition, diagnosis and classification of diabetes mellitus and its complications. Report of a WHO consultation, 1999), the Adult Treatment Panel III Report 2001 (Expert panel on detection, evaluation, and treatment of high blood cholesterol in adults. JAMA. 2001; 285:2486-97), the European Group for the Study of IR (EGIR) 1999 (Balkau B et al., Diabet Med 1999; 16:442-3), and the International Diabetes Federation (IDF) consensus on Met S.

In some embodiments, the invention is directed to treating or preventing diseases characterized by fat accumulation in the liver by administering to a mammal in need thereof a prophylactically or therapeutically effective amount of a composition comprising a compound comprising a helminth-derived glycan and/or glycoconjugate thereof (e.g., a compound comprising a Lewis antigen such as LNFPIII and SEA). In a preferred embodiment, the mammal in need of such treatment has or is at risk of developing MetS.

As reviewed in Gupta et al. (Bioscience Trends 2010; 4:204-11), risk factors for MetS include (a) a high level of triglycerides (≧150 mg/dL), (b) a low level of high density lipoprotein (HDL) cholesterol (<50 mg/dL for women and <40 mg/dL for men), (c) high blood pressure (≧130/85 mmHg), (d) high levels of fasting blood glucose (>100 mg/dL), and (e) a large waist circumference (≧35 inches for women and ≧40 inches for men). Actual values for each of the parameters should not be considered as limiting, as each of the four main criteria described supra rely on slight variations of these values. Accordingly, specific values for each of the parameters listed above should conform to values set forth in the particular criteria relied upon. Accordingly, subjects at risk of developing MetS can be readily identified based on the presence of any or combinations of the risk factors listed above.

Each of the criteria set forth by the WHO, EGIR, NCEP-ATP III, and IDF can serve as a source of direction for diagnosing individuals who have MetS. For example, the WHO criteria for MetS requires an individual to have diabetes mellitus, impaired glucose tolerance, impaired fasting glucose or insulin resistance, and at least two of the risk factors listed above. Accordingly, individuals who have MetS can be readily identified by relying on, e.g., one of the four criteria set forth above.

All publications, patents, and patent applications cited herein, whether supra or infra, are hereby incorporated by reference in their entirety.

The above disclosure generally describes the present disclosure, which is further exemplified by the following examples. These specific examples are described solely for purposes of illustration, and are not intended to limit the scope of this disclosure. Although specific targets, terms, and values have been employed herein, such targets, terms, and values will likewise be understood as exemplary and non-limiting to the scope of this disclosure.

### EXAMPLES

### Materials and Methods used in the Examples

### Animals

Male C57BL/6J mice (8-10 weeks of age) were purchased from The Jackson Laboratory (Bar Harbor, Me.). FXR α−/− mice (both in the C57BL/6J background) were obtained from The Jackson Laboratory. All animal studies were approved by the Harvard Medical Area Standing Committee on Animals.

### Treatment Regimen

Male C57BL/6J mice (8-10 weeks of age) were placed on a high-fat, high carbohydrate diet (F3282, Bio-Serv) for the duration for the experiments. After 6 weeks of the special diet, mice were intraperitoneally injected 2 times per week with 25 μg of dextran (vehicle) or 25 μg of LNFPIII conjugated with dextran (LNFPIII-dex). A second cohort was treated with 0.9% NaCl or SEA dissolved in 0.9% NaCl (25 μg/mouse/twice a week). Metabolic studies started 4 weeks after LNFPIII or SEA treatment and were conducted after 6 hrs of fasting. Animals were sacrificed at the 6^{th} week of treatment for serum and tissue collection. Most experiments were repeated in two cohorts (n=4-7/treatment). Body weight and food intake were monitored weekly.

### Measurements

Circulating levels of ALT and AST, markers of overall liver function, were measured using commercial kits as described in Liu et al. (JBC 2011; 286:1237-47).

### Histology

The Dana Farber/Harvard Cancer Center Research Pathology Cores provided all histological services and preliminary assessments by a pathologist. Briefly, tissues were collected from animals and fixed in 10% formalin (liver) for 48 hours. Fixed samples were rinsed in PBS and sent to the histology core to be paraffin embedded, sectioned, and stained with hematoxylin and eosin according to standard procedures. A staff pathologist analyzed the samples for signs of inflammation, fatty liver, fibrosis, and other pathologies associated with the liver.

### Primary Cells, Cell Culture, and Functional Assays

Primary hepatocytes were isolated as previously described (Liu et al., supra; Reilly et al., Cell Metab 2010; 12:643-53). Briefly, mice were anesthetized and perfused with 50 mL of EDTA buffer and 50 mL of perfusion buffer containing collagenase (Liberase Tm, Roche) via the portal vein. Post perfusion, hepatocytes were isolated and separated by a percoll gradient. Cells were counted and allowed to attach overnight in William's E, 5% FBS, followed by treatments for 24 hours. For de novo lipogenesis, cells were labelled with ¹⁴C-acetate and ¹⁴C-lipids were extracted with chloroform:methanol (v/v 2:1) six hours later. Samples were spun and the lipid containing layer was isolated and allowed to dry. Levels of radioactivity were then measured. β-oxidation assays were conducted as described previously (Reilly et al., supra). Briefly, cells were treated with ³H-palmitic acid for 2 hrs or 4 hrs. Levels of ³H₂O, a byproduct of lipid catabolism, in the supernatant were measured.

HepG2 cells, obtained from ATCC (#HB-8065), were cultured in Eagle's minimal essential media supplemented with non-essential amino acids, L-glutamine, sodium pyruvate, sodium bicarbonate, 10% fetal bovine serum, and antibiotics.

### Gene Expression Analyses

Relative expression levels of transcripts were determined by SYBR green-based real-time quantitative PCR (q-PCR) reactions. 36B4 levels were used for normalization. qPCR was performed with the StepOne Real-Time PCR System (Applied Biosystems). Primer sequences are shown in Table 1.

TABLE 1

| | Forward | Reverse |
|---|---|---|
| F4/80 | CTTTGGCTATGGGCTTCCAGTC | GCAAGGAGGACAGAGTTTATCGTG |
| Arg1 | CACAGTCTGGCAGTTGGAAG | GGGAGTGTTGATGTCAGTGTG |
| Mgl1 | TGAGAAAGGCTTTAAGAACTGGG | GACCACCTGTAGTGATGTGGG |
| IL1b | AATCTATACCTGTCCTGTGTAATGAAAGAC | GGT ATT GCT TGG GAT CCA CAC T |
| IL10 | CTGGACAACATACTGCTAACCG | GGGCATCACTTCTACCAGGTAA |
| IL6 | CTCTGGGAAATCGTGGAAAT | CCAGTTTGGTAGCATCCATC |
| FAS | TCCTGGAACGAGAACACGATCT | GAGACGTGTCACTCCTGGACTTG |
| ACC1 | CGCTCGTCAGGTTCTTATTG | TTTCTGCAGGTTCTCAATGC |
| ACC2 | CGCTCACCAACAGTAAGGTGG | GCTTGGCAGGGAGTTCCTC |
| SCD1 | ACGCCGACCCTCACAATTC | CAGTTTTCCGCCCTTCTCTTT |
| Srebp1c | GGAGCCATGGATTGCACATT | GCTTCCAGAGAGGAGGCCAG |
| AOX | CAGACAGAGATGGGTCATGG | ATGAACTCTTGGGTCTTGGG |
| MCAD | TTTCGAAGACGTCAGAGTGC | TGCGACTGTAGGTCTGGTTC |
| BSEP | GAAACGACGGCGTTCGTGGG | GCCATCCAGAGTCACCATGCCTT |
| PLTP | CGCCCCACGTGACCACACTAC | GGCCTTCCTGCTTCACCAGATCCA |
| OATP1 | CAGATTCAGGCACATTTACCTGGGG | TGTCTGGAGAGTGGATGTCGCCA |
| LXRa | AAGCTGGTGAGCCTCCGTACTTTG | GCGAAACAGTCACTCGTGGACATC |
| FXRa½ | CAGCCACCGGCTGTCAGGATT | GGCCTGCCGCGTGTTCTGT |
| FXRa¾ | TTGGGCACTCCCATTTACAGGCT | GGCCTGCCGCGTGTTCTGTTA |
| SHP | CGATCCTCTTCAACCCAGATG | AGGGCTCCAAGACTTCACACA |
| 36B4 | TCATCCAGCAGGTGTTTGAC | TACCCGATCTGCAGACACAC |
| Mut1 | ccagcttactaggAATTCtggcattaagaactttcc | ggaaagttcttaatgccaGAATTcctagtaagctgg |
| Mut2 | ggcattaagaacttTCTAGAtaaacatttac | gtaaatgtttaTCTAGAaagttcttaatgcc |
| Mut3 | ccgtaatgaagttaaCGCgtaaaccacacaacc | ggttgtgtggtttacGCGttaacttcattacgg |

For Western blot analyses, tissue and cell lysates were prepared in lysis buffer (20 mM Tris-HCl, 100 mM NaCl, 1 mM EDTA, NP-40, glycerol, PMSF, and DTT) in the presence of protease and phosphatase inhibitors. The four FXRα (NR1H4) isoforms were based on previous reports, which were originally designated as FXRα1/α2 and FXRβ1/β2 (Huber et al., Gene 2002; 290:35-43; Zhang et al., JBC 2003; 278:104-10). The latter were renamed FXRα3/α4 to avoid confusion with rodent FXRβ (NR1H5) (Lefebvre et al., Physiol Rev 2009; 89:147-91).

### Luciferase Reporter Assays

The upstream promoter 1 and downstream promoter 2 regions of the human FXRα gene were cloned into the pGL3-basic luciferase reporter construct and transfected into HepG2 cells in a 96-well format or primary hepatocytes in a 24-well format using a luciferase system (Promega). A β-galactosidase reporter construct was used as a transfection control. Promoters 1 and 2 of the human FXRα gene were PCR amplified and cloned into the pGL3-basic luciferase reporter (Promega). Point mutations were introduced using the site-directed mutagenesis kit (Stratagene). Primers for generating specific mutations are listed in Table 1.

### Statistical Analysis

Statistical analysis was performed using Student's t test (two-tailed). Values are presented as mean±SEM. P<0.05 was considered significant.

### Example 1

### Treatment of High Fat Diet-Induced Steatosis and Preservation of Liver Function by LNFPIII

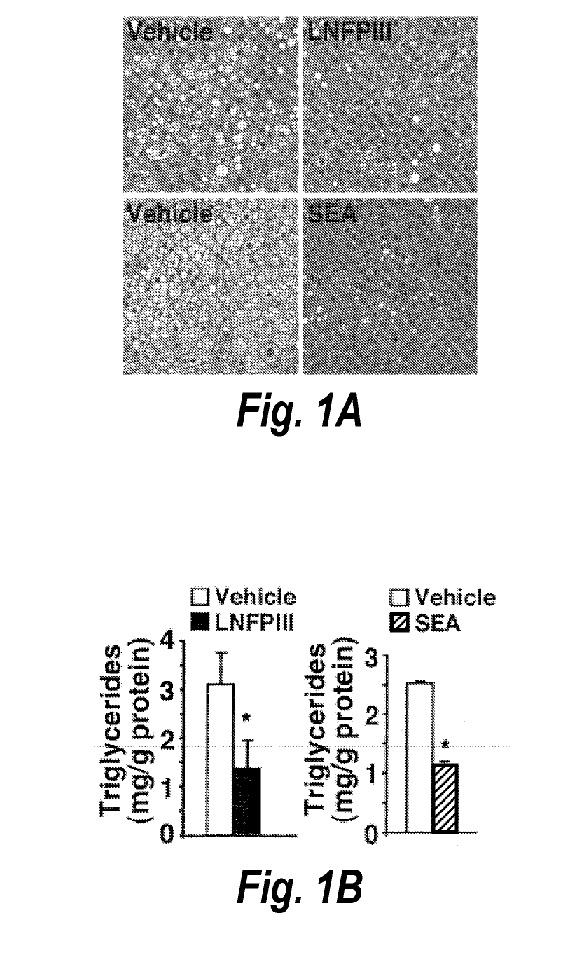

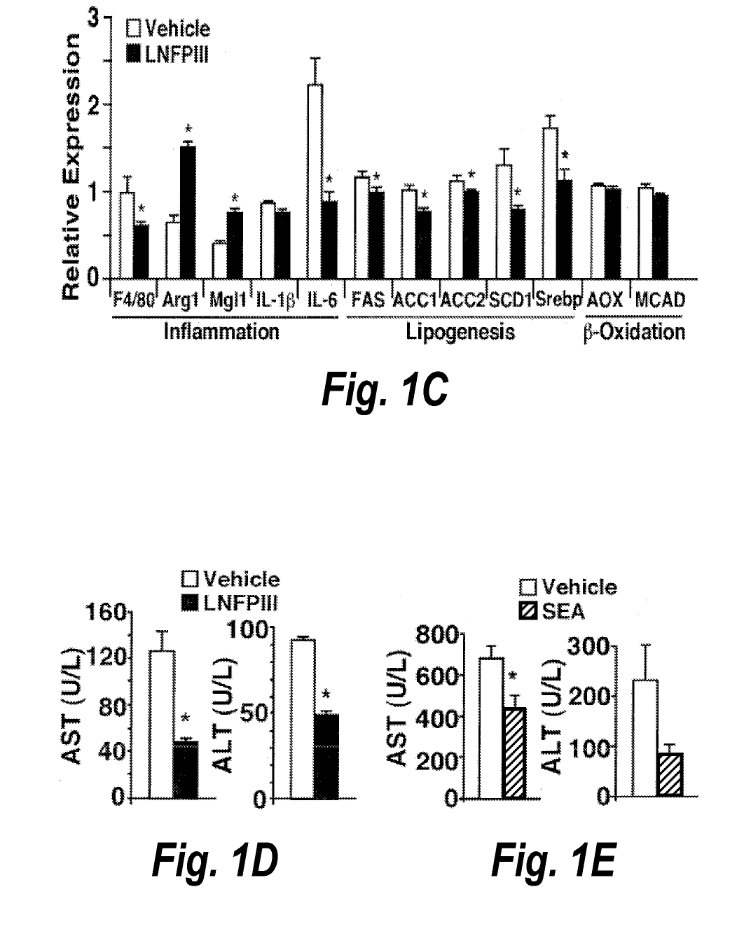

In this example, the ability of LNFPIII or SEA to prevent high fat diet-induced steatosis and preserve liver function was examined. Mice were injected with either vehicle (dextran, 25 μg), LNFPIII conjugated to dextran (25 μg), or SEA twice a week (n=5 for each group) as described in the Methods section. Histological assessment revealed less high fat diet-induced hepatic lipid accumulation in livers of LNFPIII- or SEA-treated mice compared to vehicle-treated mice (FIG. 1a). Furthermore, there were significantly lower levels of triglycerides in livers of LNFP- or SEA-treated mice compared to vehicle treated mice (*p<0.05; FIG. 1b). Similar results are expected with other animal models of FLD including, e.g., virus-induced FLD (e.g., HIV or hepatitis virus-induced).

To determine whether the decreased lipid accumulation and lower triglyceride levels in the liver could be attributed to differences in inflammation, lipogenic gene expression, or β-oxidation, liver tissue was harvested and mRNA was extracted and processed for real time q-PCR. Mice treated with LNFPIII exhibited significantly lower levels of F4/80 markers and IL-6 in liver tissue, but significantly higher levels of M2 markers (Arg1 and Mgl1) compared to vehicle treated mice (*p<0.05; FIG. 1c). Levels of IL-1β were unchanged. These results suggest that LNFPIII reduced the infiltration of pro-inflammatory macrophages into hepatic tissue while increasing the number of M2 macrophages. Surprisingly, expression of lipogenic genes, including fatty acid synthase (FAS), acetyl-CoA carboxylate 1/2 (ACC1/2), stearoyl-CoA desaturase 1 (SCD1), and sterol regulatory element-binding protein 1c (SREBP-1c) were significantly reduced in livers of LNFPIII-treated mice compared to vehicle-treated mice (*p<0.05; FIG. 1c). These results are consistent with the notion that LNFPIII reduces inflammation and fat accumulation in the liver.

To determine whether overall liver function was affected by LNFPIII treatment, circulating levels of AST and ALT were also examined. Compared to vehicle-treated mice on a high fat diet, LNFPIII-treated mice showed significantly reduced levels of AST and ALT (*p<0.05; FIG. 1d). SEA exhibited similar protective effects (*p<0.05; FIG. 1e).

Similar results can be expected with combination therapy involving administration of multiple distinct helminth-derived glycan species (e.g., LNFPIII, LnNT, LDN, and LDNF) individually, or alternatively, multiple distinct helminth-derived glycan and/or glycoconjugate species can be conjugated to the same carrier molecule.

Taken together, these results indicate that LNFPIII suppresses liver inflammation which accompanies hepatic fat accumulation and prevents ectopic fat accumulation in the liver.

### Example 2

### LNFP III-Mediated Suppression of Lipogenesis Through FXRα Activation in the Liver

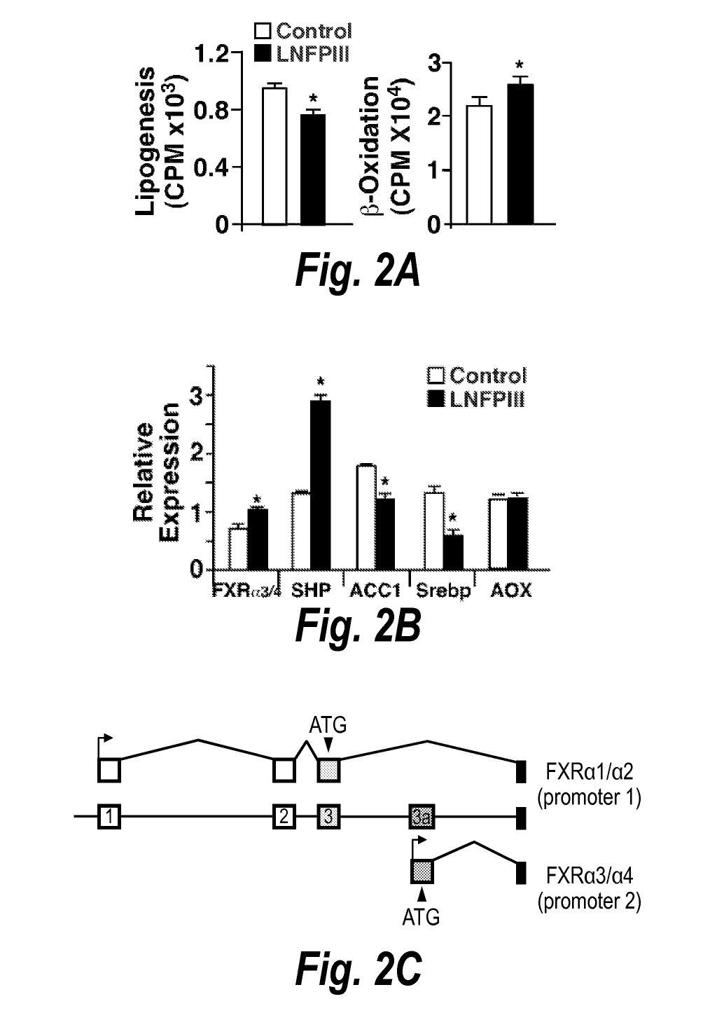

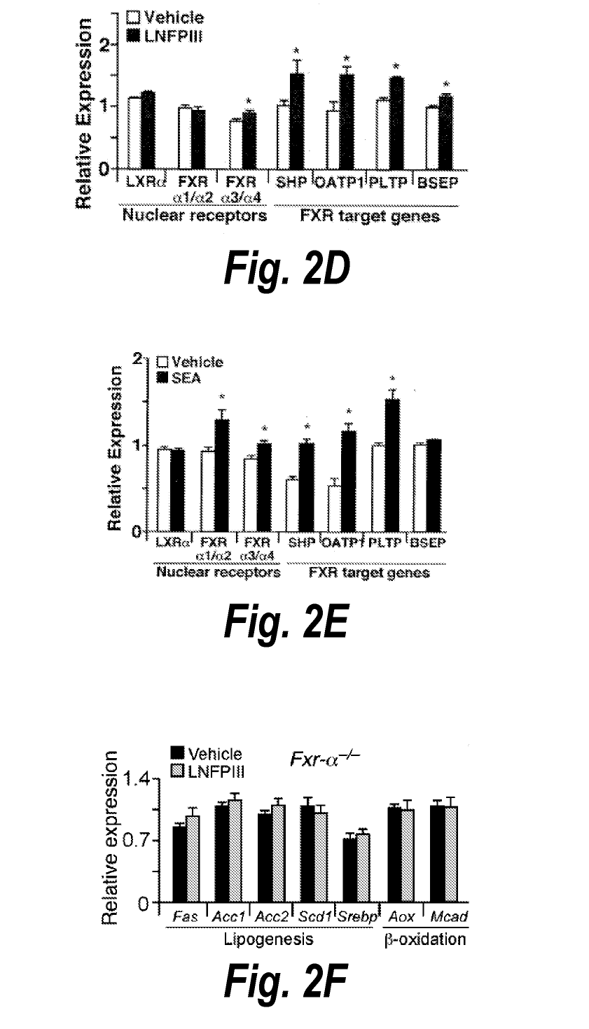

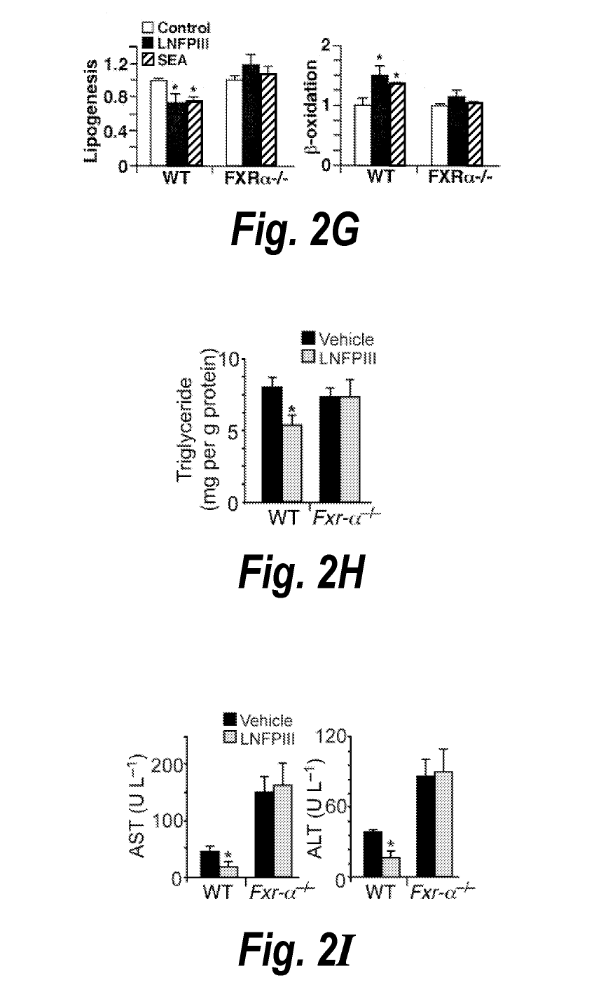

Based on the reduced lipogenic gene expression profile seen in the liver (see Example 1), the ability of LNFP III to suppress lipogenesis was assessed. To this end, primary mouse hepatocytes were isolated and subjected to de novo lipogenesis assays upon treatment with LNFP III (20 μg/ml) or vehicle (dextran). LNFPIII significantly suppressed lipogenesis and increased fatty acid β-oxidation in hepatocytes compared to vehicle treated cells (*p<0.05; FIG. 2a). Given that LNFP III treatment did not affect β-oxidation gene expression (FIGS. 1c and 2b), the increased fat burning was likely secondary to decreased fatty acid synthesis.

In order to gain mechanistic insight into how LNFPIII was suppressing lipogenesis, SREBP-1c was focused on given its role as a master lipogenic transcription factor and its downregulation in liver tissue of mice treated with LNFPIII (FIG. 1c). SREBP-1c expression and transcriptional activity are controlled by a network of nuclear receptor signalling pathways, notably, the positive regulator LXRα (NR1H3) (Joseph et al., JBC 2002; 277:11019-25; Repa et al., Genes Dev 2000; 14:2819-30) and negative regulators FXRα (NR1H4) and its target SHP (NR0B2) (Watanabe et al., J Clin Invest 2004; 113:1408-18). FXRα consists of two major 5′ regulatory regions, with the upstream and downstream promoters driving the expression of FXRα1/α2 and FXRα3/α4 isoforms, respectively (Lefebvre et al., Physiol Rev 2009; 89:147-91), (FIG. 2c). The only difference between FXRα1/α2 (or FXRα3/α4) is a 4 amino acid insertion in FXRα1 (or FXRα3).

Transcripts of the FXRα3/α4 isoform, as well as several known FXR target genes, including SHP, organic anion-transporting polypeptides (OATP), phospholipid transfer protein (PLTP), and bile salt efflux pump (BSEP), were significantly upregulated in primary hepatocytes treated with LNFPIII for 24 hours compared to vehicle-treated hepatocytes by real-time q-PCR (*p<0.05; FIG. 2d); however, FXRα1/α2 and LXRα mRNA expression remained unchanged. Moreover, LNFPIII treatment of primary hepatocytes was sufficient to significantly increase FXRα3/α4 and SHP mRNA expression as well as significantly suppress SREBP-1c and ACC1 levels (*p<0.05; FIG. 2b). Similar effects were obtained with SEA treatment, except that SEA appeared to significantly upregulate both FXRα1/α2 and FXRα3/α4 (*p<0.05; FIG. 2e).

Consistent with FXRα mediating the downregulation of genes involved in lipogenesis, LNFPIII treatment did not reduce the mRNA expression of Fas, Acc1, Acc2, Scd1, and Srebp in FXRα^{-/-} mice (FIG. 2f). Furthermore, to determine whether FXRα mediated the effects of LNFPIII on lipogenesis and fatty acid oxidation, primary hepatocytes were isolated from wild type or FXRα^{-/-} mice, cultured, and tested for de novo lipogenesis and fatty acid oxidation. Consistent with FXRα mediating the effects of LNFPIII, LNFPIII and SEA failed to inhibit lipogenesis and enhance fatty acid oxidation in FXRα^{-/-} hepatocytes (*p<0.05; FIG. 2g). As shown in FIGS. 2h and 2i (*p<0.05), the reduction in hepatic triglyceride content and serum AST and ALT concentrations by LNFPIII treatment also required FXRα expression. These data suggest that LNFPIII directly targets FXRα to modulate hepatic lipid metabolism.

### Example 3

### Signaling Pathways that Link LNFPIII and FXRα

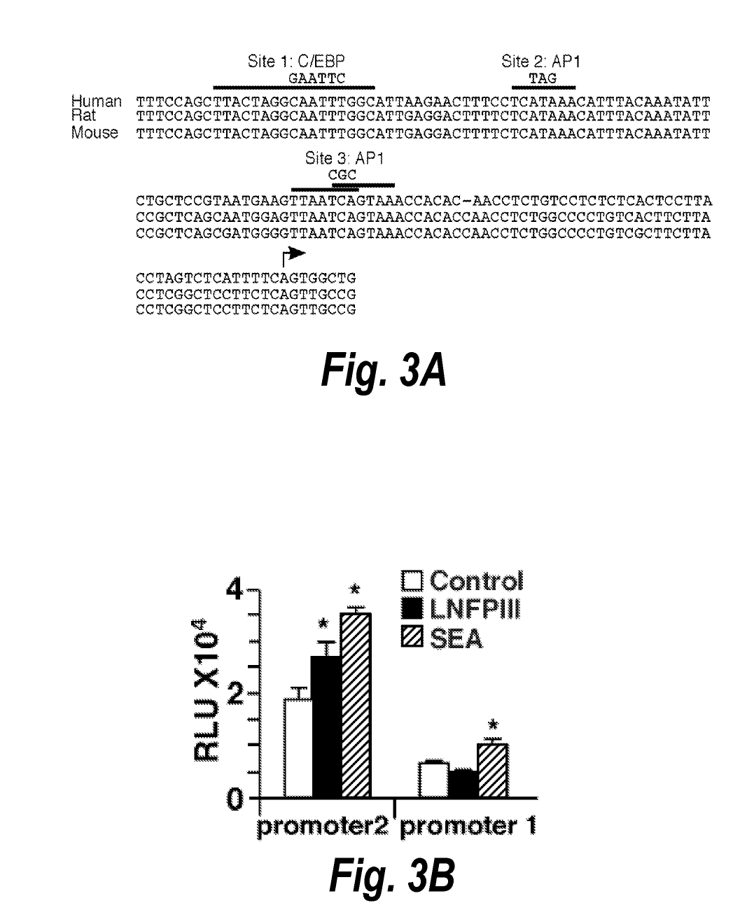

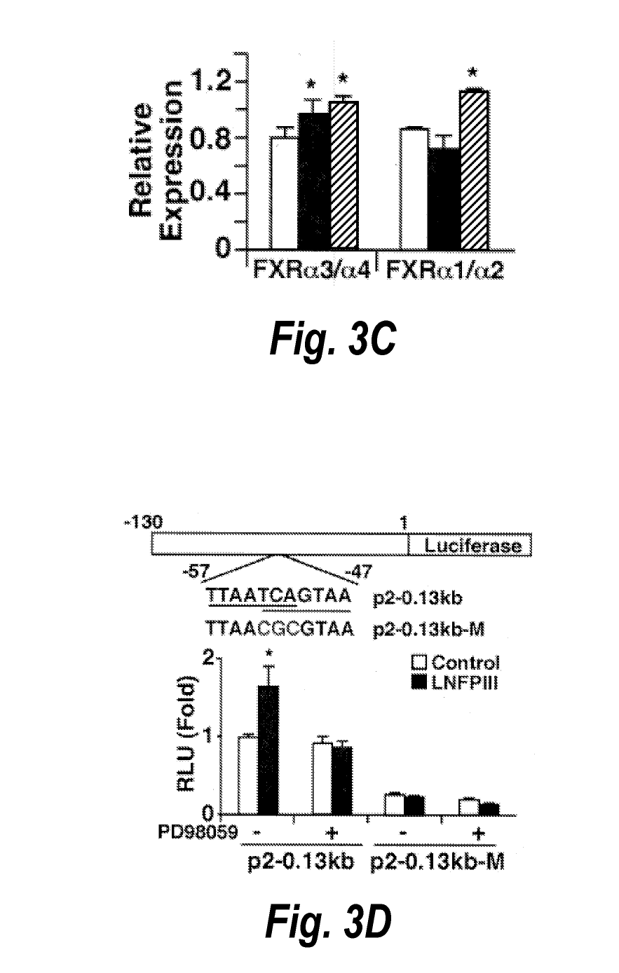

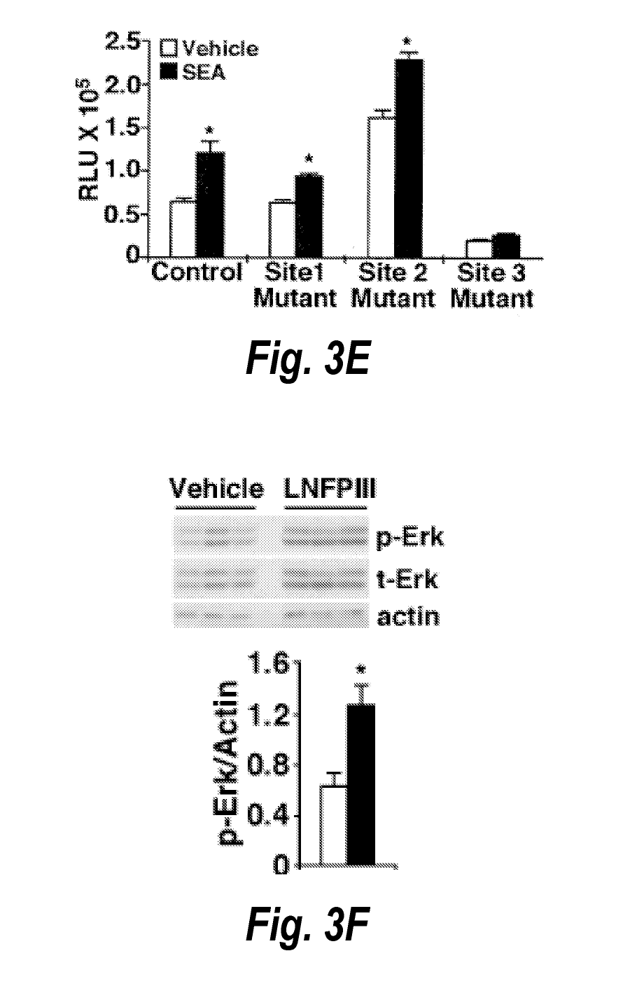

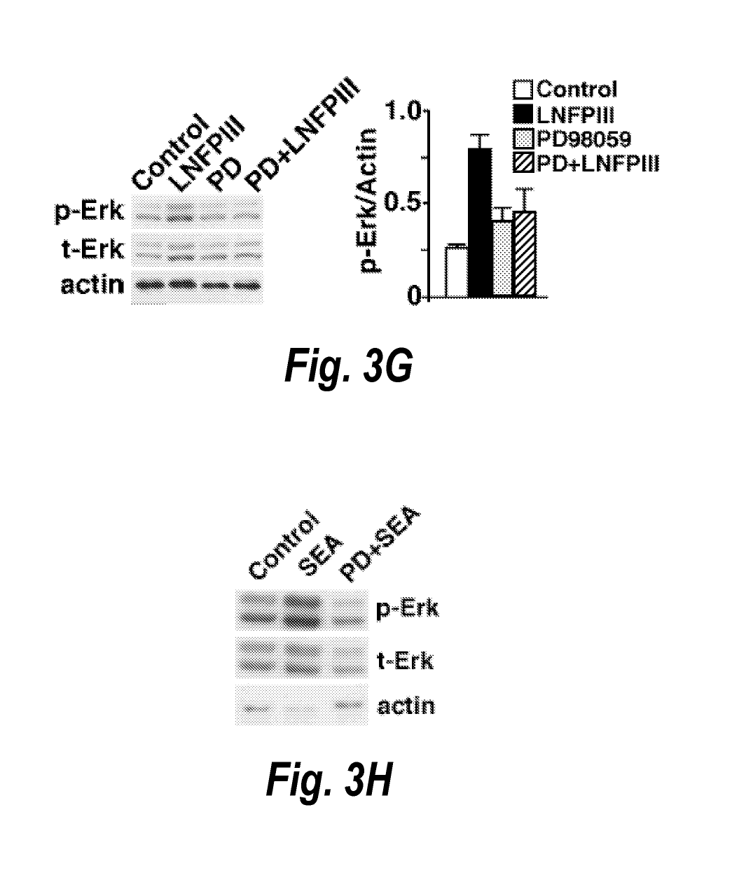

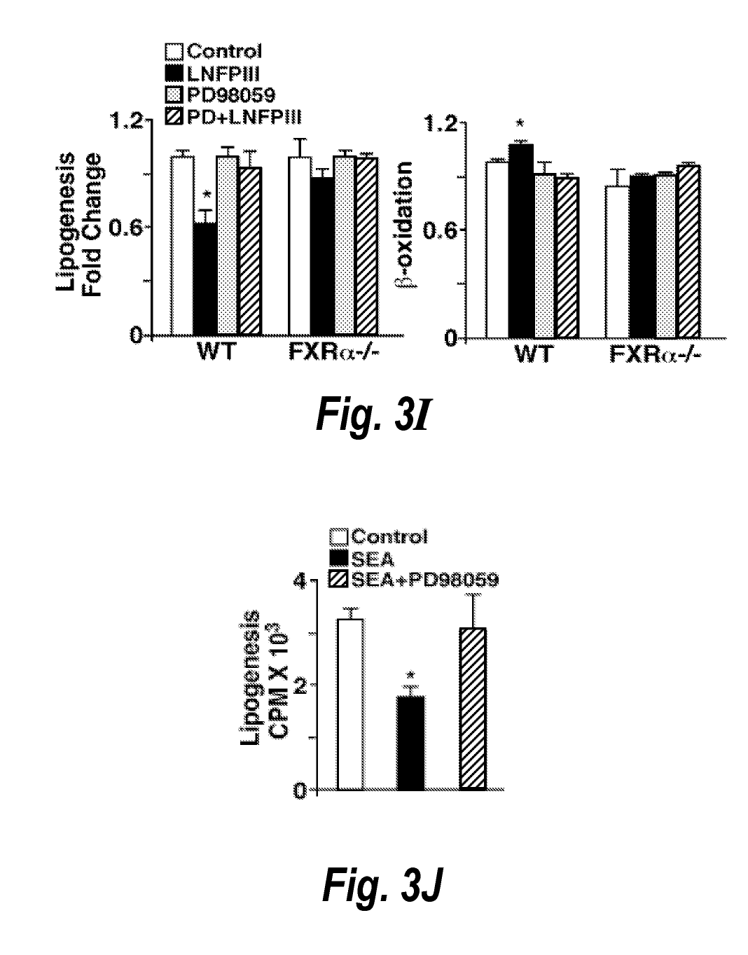

Both human and mouse FXRα contain two major promoters (FIG. 2c). FIG. 2c shows the genomic structure showing alternative promoter usage to drive the expression of human FXRα1/α2 (promoter 1) and FXRα3/α4 (promoter 2). Sequence comparison reveals high conservation in the 5′ regulatory sequences between mouse, rat, and human FXRα genes (FIG. 3a). To examine the signalling pathways through which LNFPIII regulates FXRα activity, the activities of luciferase reporters driven by upstream (promoter 1) or downstream (promoter 2) regulatory regions were examined in HepG2 cells (human hepatoma cells). Reporters driven by a 2 kb fragment of human FXRα promoter 1 or a 0.13 kb fragment of human FXRα promoter 2 were transfected into HepG2 cells. Twenty-four hours after transfection, cells were treated with or without LNFPIII (20 μg/ml) or SEA (2 μg/ml) for an additional 24 hours. When HepG2 cells were transfected with promoter 1- or promoter 2-luciferase reporter constructs, only promoter 2 was significantly activated in response to LNFPIII treatment, whereas both promoters were significantly activated by SEA (*p<0.05; FIG. 3b). Consistent with this, only FXRα3/α4 expression was significantly increased by LNFPIII treatment (20 μg/ml), whereas both FXRα1/α2 and FXRα3/α4 were induced by SEA (2 μg/ml) in primary hepatocytes (*p<0.05; FIG. 3c).

To gain insight into the region of promoter 2 responsible for promoter activation, the minimal LNFPIII responsive region was determined through a series of promoter deletion mutants. The minimal LNFPIII responsive region is highly conserved between human, rat, and mouse, and mapped to approximately 130 bp upstream of the transcriptional start site and contained consensus binding sites for C/EBP and AP1 (FIG. 3a). With respect to the AP-1 sites, there were 3 potential binding sites; one independent site (i.e., Site 2), and another site which had two overlapping AP-1 sites (i.e., Site 3) (FIG. 3a). Site-directed mutagenesis demonstrated that the two overlapping AP-1 sites were required for the activation of the FXRα promoter by LNFPIII (FIG. 3d). Similar results were obtained with SEA (FIG. 3e).

Previous studies have demonstrated that in dendritic cells, LNFPIII signals through Erk to induce the activation of c-fos, which dimerizes with c-jun to form AP1 (Ham et al., Immunol Rev 2009; 230:247-57; Dillon et al., J Immunol 2004; 172:4733-43). Consistent with this and the finding that AP-1 binding site was required for LNFPIII-mediated activation of the FXRα promoter, Erk phosphorylation was increased in LNFPIII treated liver (FIG. 3f) and SEA treated primary hepatocytes (FIG. 3h). Indeed, when a chemical MEK inhibitor, PD98059 (10 μM for one hour prior to overnight LNFPIII treatment), was used to block Erk activation, it completely abolished the responsiveness of the FXRα promoter 2 to LNFPIII in HepG2 cells (FIG. 3d), the activation of Erk in response to LNFPIII (FIG. 3g), and the ability of LNFPIII to suppress lipid synthesis and increase β-oxidation in primary hepatocytes (FIG. 3i). Notably, LNFPIII-mediated suppression of lipogenesis and increase in β-oxidation requires FXRα, as evidenced by the inability to suppress lipogenesis or increase β-oxidation in FXRα^{-/-} hepatocytes. Similar results were obtained with SEA (*p<0.05; FIGS. 3h and 3j). These findings collectively suggest that LNFPIII is a direct effector of hepatic lipogenesis through the Erk-c-fos/AP-1-FXRα axis.

### Example 4

### Effects of LNnT on Hepatic Lipogenesis and FXRα Activation

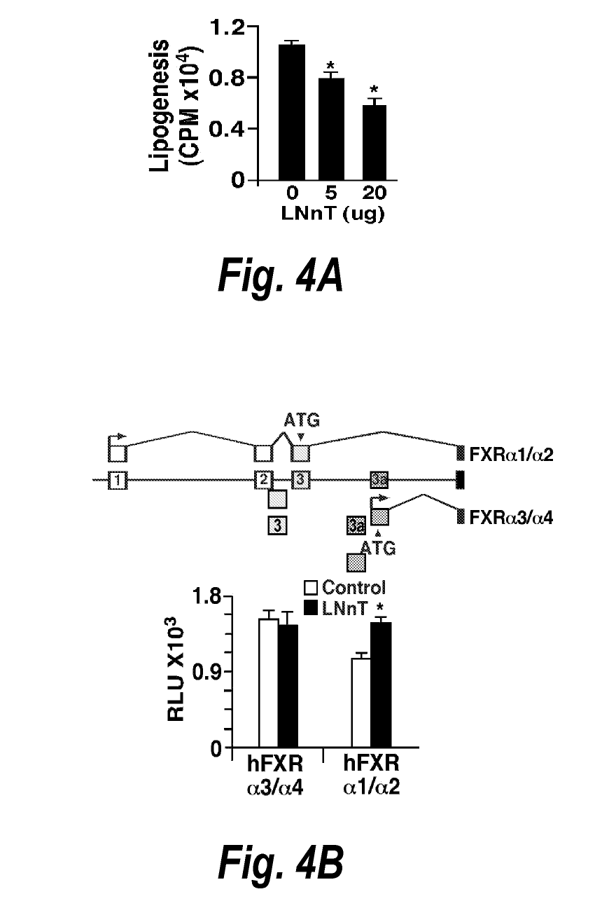

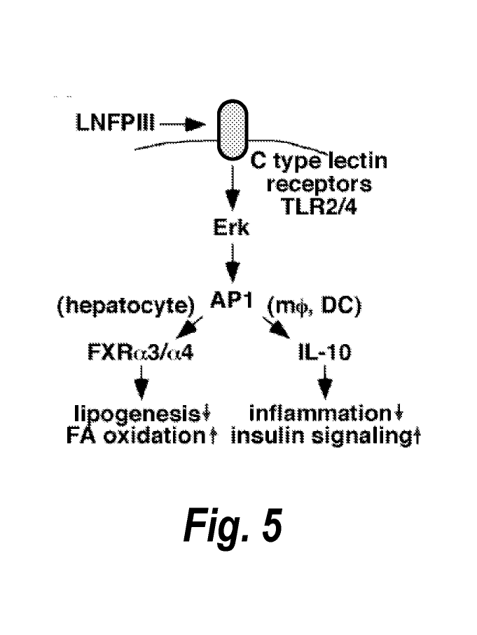

Consistent with the inhibitory effects of LNFPIII and SEA on hepatic lipogenesis, LNnT (a non-Lewis antigen) blocked lipogenesis in cultured primary hepatocytes in a dose-dependent manner (FIG. 4a; *p<0.05). Furthermore, LNnT significantly increased the activity of the FXRα1/α2 promoter in a luciferase assay performed as described in Example 3 (FIG. 4b; *p<0.05). These findings indicate that glycans other than LNFPIII can suppress hepatic lipogenesis through FXRα activation.

### Example 5

### Prophylactic and Long-Term Treatment of Fatty Liver Disease

For prophylactic treatment of FLD, animals are injected with one or more helminth-derived glycans or glycoconjugates (e.g., LNFPIII, SEA, LnNT, LDN, LDNF, or a combination of glycans) prior to feeding mice with a high-fat diet. Effects of these glycans on prophylaxis of hepatosteatosis are determined using the assays described in Examples 1-3. It is expected that mice injected with the various glycans will not develop hepatic steatosis or fatty liver disease.

To assess the long-term effects of helminth-derived glycans and/or glycoconjugates thereof on serum free fatty acid and triglyceride levels, as well as effects on other tissues, animals are treated twice/week for 12-16 weeks, or longer, and subjected to the assays described supra. Reduced levels of serum free fatty acid and triglycerides are expected, as well as reduced peripheral tissue lipid accumulation, with administration of helminth-derived glycans and/or glycoconjugates thereof. Serum triglyceride levels can also be regularly monitored to follow the development or treatment of FLD.

While the disclosure has been described in each of its various embodiments, it is expected that certain modifications thereto may be undertaken and effected by the person skilled in the art without departing from the true spirit and scope of the disclosure, as set forth in the previous description and as further embodied in the following claims. The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims. It is further to be understood that all values are approximate, and are provided for description. Those skilled in the art will recognize, or be able to ascertain using no more than routine experimentation, many equivalents to the specific embodiments of the invention described herein. Such equivalents are intended to be encompassed by the following claims.

1. A method of treating or preventing a disease associated with fat accumulation in the liver, comprising administering to a mammal in need thereof a therapeutically effective amount of a compound comprising a helminth-derived glycan and/or glycoconjugate thereof.

2-3. (canceled)

4. The method of claim 1, wherein the compound comprises at least two a helminth-derived glycan and/or glycoconjugate thereof.

5. The method of claim 1, wherein the compound comprises a helminth-derived glycan and/or glycoconjugate thereof selected from the group consisting of LNFPIII, LNnT, LDN, and LDNF.

6. The method of claim 1, wherein the helminth-derived glycan and/or glycoconjugate thereof comprises a Lewis antigen.

7-8. (canceled)

9. The method of claim 1, wherein the compound comprises SEA.

10. The method of claim 1, wherein the disease is selected from the group consisting of fatty liver disease, hepatic steatosis, steatohepatitis, metabolic syndrome or a combination thereof.

11-18. (canceled)

19. The method of claim 1, wherein at least one helminth-derived glycan and/or glycoconjugate thereof is conjugated to the carrier molecule.

20. (canceled)

21. The method of claim 19, wherein the molecular weight of the conjugate of helminth-derived glycan and/or glycoconjugate thereof and carrier molecule is about 5,000 to 100,000 daltons.

22. (canceled)

23. The method of claim 21, wherein the conjugate has 2-200 helminth-derived glycan- and/or glycoconjugate-containing oligosaccharide molecules per carrier molecule.

24-25. (canceled)

26. The method of claim 19, wherein the carrier molecule is selected from the group consisting of a carbohydrate polymer, a protein, polyacrylamide, or a combination of one or more thereof.

27. The method of claim 26, wherein the carbohydrate polymer is dextran.

28-30. (canceled)

31. The method of claim 1, wherein the compound is administered parenterally, intraperitoneally, intravenously or orally.

32-34. (canceled)

35. The method of claim 1, wherein administration of the compound comprising a helminth-derived glycan and/or glycoconjugate thereof has one or more of the following effects:
(a) reduces triglyceride levels in the liver;
(b) suppresses lipogenesis in the liver;
(c) increases production of FXRα in hepatocytes; and
(d) reduces production of SREBP-1c in hepatocytes.

36-39. (canceled)

40. A method of reducing lipogenesis in a hepatocyte, the method comprising contacting the hepatocyte with a sufficient amount of a compound comprising a helminth-derived glycan and/or glycoconjugate thereof.

41-49. (canceled)

50. The method of claim 40, wherein the compound is administered to a subject in need thereof such that hepatic lipogenesis is inhibited.

51-73. (canceled)

74. A method of increasing the production of FXRα in a cell, comprising contacting the cell with a compound comprising a helminth-derived glycan and/or glycoconjugate thereof.

75-92. (canceled)

93. A method of increasing the production of a gene product that is transcriptionally induced by FXRα, comprising contacting the hepatocyte with a compound comprising a helminth-derived glycan and/or glycoconjugate thereof.

94-109. (canceled)

110. The method of claim 93, wherein the gene product is selected from the group consisting of SHP, OATP1, PLTP, and BSEP.

111. A method of increasing activation of the Erk-c-fos/AP1-FXRα pathway in a hepatocyte, comprising contacting the hepatocyte with a compound comprising a helminth-derived glycan and/or glycoconjugate thereof.

112-128. (canceled)

129. A pharmaceutical composition comprising a helminth-derived glycan and/or glycoconjugate thereof in an amount sufficient to treat a disease associated with fat accumulation in the liver.
